(19) United States
(12) Patent Application Publication
(10) Pub. No.: US 2004/0176459 A1
(43) Pub. Date: Sep. 9, 2004

(54) Prodrugs of excitatory amino acids

(76) Inventors: Ana Belen Bueno Melendo, Madrid (ES); Alfonso De Dios, Madrid (ES); Carmen Dominguez-Fernandez, Madrid (ES); Luisa Maria Martin-Cabrejas, Madrid (ES); Jose Alfredo Martin, Madrid (ES); Carlos Montero Salgado, Madrid (ES); Concepcion Pedregal-Tercero, Madrid (ES); Rafael Ferritto Crespo, Madrid (ES); Marc Francis Herin, Perwez (BE); Maria Angeles Martinez-Grau, Madrid (ES); Steven Marc Massey, Indianapolis, IN (US); James Allen Monn, Indianapolis, IN (US); Matthew John Valli, Zionsville, IN (US)

Correspondence Address: Arvie J Anderson, Eli Lilly & Company, Patent Division, PO Box 6288, Indianapolis, IN, 46206-6288, US

(21) Appl. No.: 10/480,517

(22) PCT Filed: Jul. 2, 2002
(86) PCT No.: PCT/US02/19825

(30) Foreign Application Priority Data
Jul. 9, 2001 (GB) 01500175.3
Nov. 23, 2001 (GB) 01500275.1

(51) Int. Cl. A61K 31/198; C07C 229/28

(52) U.S. Cl. 514/566; 562/501

(57) ABSTRACT

This invention relates to synthetic excitatory amino acid prodrugs and processes for their preparation. The invention further relates to methods of using, and pharmaceutical compositions comprising, the compounds for the treatment of neurological disorders and psychiatric disorders.

This invention relates to synthetic excitatory amino acid prodrugs (and their pharmaceutically acceptable salts) and processes for their preparation. The invention further relates to methods of using, and pharmaceutical compositions comprising, the compounds for the treatment of neurological disorders and psychiatric disorders.

Treatment of neurological or psychiatric disorders, such as anxiety disorder, have been linked to selective activation of metabotropic excitatory amino acid receptors such as (+)-2-aminobicyclo[3.1.0]hexane-2,6-dicarboxylic acid, also known as LY354740, which is disclosed in U.S. Pat. No. 5,750,566 (the '566 patent) issued May 12, 1998 is an active mGlu2 receptor agonist. CNS Drug Reviews, 5, pgs. 1-12 (1999).

The present invention provides for a prodrug form of LY354740 which enhances the oral exposure of LY354740. The present invention also provides for prodrug forms of other compounds which possess improved oral exposure. Compounds of the present invention represent an improved approach for maintaining LY354740-like safety and efficacy in humans with increased oral bioavailability. Preclinical studies with compounds of the present invention, has shown greatly enhanced oral exposure of the parent compound.

Accordingly, the present invention provides a compound of the formula I

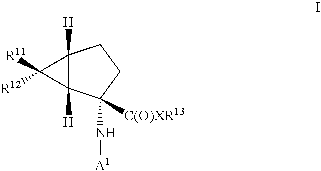

wherein

R¹¹ is C(O)YR¹⁴ and R¹² is hydrogen or fluoro; or R¹¹ is hydrogen or fluoro and R¹² is C(O)YR¹⁴;

R¹³ and R¹⁴ are, independently, hydrogen, (1-10C)alkyl, (2-4C)alkenyl, (2-4C)alkynyl, or aryl;

A¹ is hydrogen or an amino acyl bonded through the carbonyl to form an amine terminus;

X and Y are, independently, O or A²;

A² is an amino acyl bonded through the amine to form a carboxlyate terminus;

provided when X is O, Y is not O;

or a pharmaceutically acceptable salt thereof.

In one embodiment, this invention provides methods of treating neurological or psychiatric disorders, comprising: administering to a patient in need thereof an effective amount of a compound of Formula I, or a pharmaceutically acceptable salt thereof. That is, the present invention provides for the use of a compound of Formula I, or a pharmaceutical composition thereof, for the treatment of psychiatric or neurological disorders.

In another aspect, the present invention provides for the use of a compound of Formula I, or a pharmaceutically acceptable salt thereof, in the manufacture of a medicament for treating neurological or psychiatric disorders. Thus, the present invention provides for use of a compound of Formula I, or a pharmaceutically acceptable salt thereof, in the manufacture of a medicament for the treatment of neurological or psychiatric disorders.

Compounds of the invention have been found to be useful prodrugs for selective agonists of metabotropic glutamate receptors and are therefore useful in the treatment of diseases of the central nervous system such as neurological diseases, for example neurodegenerative diseases, and as antipsychotic, anxiolytic, drug-withdrawal, antidepressant, anticonvulsant, analgesic and anti-emetic agents.

It will be appreciated that the compounds of formula (I) contain at least four asymmetric carbon atoms, three being in the cyclopropane ring and one being at the a-carbon of the amino acid group within the cyclopentane ring. Additional asymmetric carbons may be present in the generic radicals as defined. Accordingly, the compounds of the invention may exist in and be isolated in enantiomerically pure form, in racemic form, or in a diastereoisomeric mixture.

The amino acid moiety within the cyclopentane ring preferably has the natural amino acid configuration, i.e. the L-configuration relating to D-glyceraldehyde.

The present invention includes pharmaceutically acceptable salts of the compound of formula I. These salts can exist in conjunction with the acidic or basic portion of the molecule and can exist as acid addition, primary, secondary, tertiary, or quaternary ammonium, alkali metal, or alkaline earth metal salts. Generally, the acid addition salts are prepared by the reaction of an acid with a compound of formula I. The alkali metal and alkaline earth metal salts are generally prepared by the reaction of the hydroxide form of the desired metal salt with a compound of formula I.

Acids commonly employed to form such salts include inorganic acids, for example hydrochloric, hydrobromic, nitric, sulphuric or phoshoric acids, or with organic acids, such as organic carboxylic acids, for example, glycollic, maleic, hydroxymaleic, fumaric, malic, tartaric, citric, salicyclic, o-acetoxybenzoic, or organic sulphonic, 2-hydroxyethane sulphonic, toluene-p-sulphonic, methane-sulfonic or naphthalene-2-sulphonic acid.

In addition to pharmaceutically-acceptable salts, other salts are included in the invention. They may serve as intermediates in the purification of compounds or in the preparation of other, for example pharmaceutically-acceptable, acid addition salts, or are useful for identification, characterization or purification.

As shown in Scheme 1 below, compounds of formula I are enzymatically or hydrolyticly converted in vivo to form compounds of formula II, where Y is O, and R¹³ and R¹⁴ are both hydrogens (a di-acid).

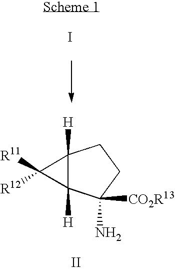

A variety of physiological functions have been shown to be subject to influence by excessive or inappropriate stimulation of excitatory amino acid transmission. The compounds of formula I the present invention are believed to have the ability to treat a variety of neorological disorders in mammals associated with this condition, including acute neurological disorder such as cerebral deficits subsequent to cardiac bypass surgery and grafting, stroke, cerebral ischemia, spinal cord trauma, head trauma, perinatal hypoxia, cardiac arrest, and hypoglycemic neuronal damage. The compounds of formula I are believed to have the ability to treat a variety of chronic neurological disorders, such as Alzheimer's disease, Huntington's Chorea, amyotrophic lateral sclerosis, AIDS-induced dementia, ocular damage and retinopathy, cognitive disorders, and idiopathic and drug-induced Parkinson's. The present invention also provides methods for treating these disorders which comprises administering to a patient in need thereof an effective amount of a compound of formula I or a pharmaceutically acceptable salt thereof.

Compounds of formula I of the present invention are also believed to have the ability to treat a variety of other neurological disorders in patients that are associated with glutamate dysfunction, including muscular spasms, convulsions, migraine headaches, urinary incontinence, psychosis, (such as schizophrenia), drug tolerance and withdrawal (such as nicotine, opiates and benzodiazepines), anxiety and related disorders, premenstural dysphoric disorder (PDD), emesis, brain edema, chronic pain, and tardive dyskinesia. The compounds of formula I are also useful as antidepressant and analgesic agents. Therefore, the present invention also provides methods for treating these disorders which comprise administering to a patient in need thereof an effective amount of the compound of formula I, or a pharmaceutically acceptable salt thereof.

A compound of formula I may be made by a process which is analogous to one known in the chemical art for the production of structurally analogous heterocyclic compounds or by a novel process described herein. Such processes and intermediates useful for the manufacture of a compound of formula I as defined above are provided as further features of the invention and are illustrated by the following procedures in which, unless otherwise specified, the meanings of the generic radicals are as defined above.

(A) For a compound of formula I in which A¹ is hydrogen and X is A², deprotecting the amine group of a compound of formula III

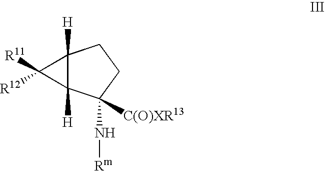

in which R^{m} is an amine-protecting group as descibed in General Procedures 6-8.

(B) For a compound of formula I in which R¹¹ is a carboxy and X is A², deprotecting the amine and carboxy groups of a compound of formula III in which R^{m} is an amine-protecting group and R¹⁴ is a carboxy protecting group as described in General Procedure 9.

(C) For a compound of formula I in which Y is A² and X is O and R¹³ is hydrogen, deprotecting and ring-opening a compound of formula IV as described in General Procedure 6.

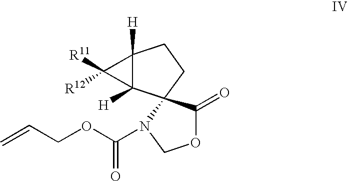

The term “amine-protecting group,” as used herein, refers to those groups intended to protect or block the amine group against undesirable reactions during synthetic procedures. Choice of the suitable amine protecting group used will depend upon the conditions that will be employed in subsequent reaction steps wherein protection is required, as is well within the knowledge of one of ordinary skill in the art. Commonly used amine protecting groups are disclosed in T. W. Greene and P. G. M. Wuts, Protective Groups In Organic Synthesis, 3rd Ed. (John Wiley & Sons, New York (1999)). Suitable amine protecting groups comprise acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, alpha-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylothoxycarbonyl, alpha, alpha-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; alkyl groups such as benzyl, triphenylmethyl, benzyloxymethyl and the like; and silyl groups such as trimethylsilyl and the like. Preferred suitable amine protecting groups are acetyl, methyloxycarbonyl, benzoyl, pivaloyl, allyloxycarbonyl, t-butylacetyl, benzyl, t-butyloxycarbonyl (Boc) and benzyloxycarbonyl (Cbz). The amine protecting group is decomposed by using a conventional procedure which does not affect another portion of the molecule.

The term “carboxy-protecting groupn as used herein refers to one of the ester derivatives of the carboxylic acid group commonly employed to block or protect the carboxylic acid group while reactions are carried out on other functional groups of the compound. Particular values include, for example, methyl, ethyl, tert-butyl, benzyl, methoxymethyl, trimethylsilyl, allyl, and the like. Further examples of such groups may be found in T. W. Greene and P. G. M. Wuts, Protecting Groups in Organic Synthesis, 3rd. Ed. (John Wiley & Sons, N.Y. (1999)). Preferred carboxy protecting groups are methyl and allyl. The ester is decomposed by using a conventional procedure which does not affect another portion of the molecule.

Whereafter, for any of the above procedures, when a pharmaceutically acceptable salt of a compound of formula I is required, it is obtained by reacting the acid of formula I with a physiologically acceptable base or by reacting a basic compound of formula I with a physiologically acceptable acid or by any other conventional procedure.

The term “(1-10C)alkyl” represents a straight, branched, or cyclic alkyl chain having from one to ten carbon atoms. Typical straight or branched (1-10C)alkyl groups include methyl, ethyl, n-propyl, isopropyl, n-butyl, isobutyl, sec-butyl, t-butyl, n-pentyl, isopentyl, neopentyl, n-hexyl, 2-methylpentyl, 3-methylpentyl, 4-methylpentyl, 2,2-dimethylbutyl, 2,3-dimethylbutyl, 3,3-dimethylbutyl, heptyl, n-octyl, 2,2-dimethylhexyl, 2,5-dimethylhexyl, 2-methylheptyl, 4-methylheptyl, 2,2,4-trimethylpentyl, 2,3,4-trimethylpentyl, nonyl, 3,5,5-trimethylhexyl, decyl, 3,7-dimethyloctyl, and the like. Typical cyclic alkyl groups include cyclopropyl, cyclobutyl, cyclopentyl, cyclohexyl, cycloheptyl, and the like. The term “(1-10C)alkyl” includes within it the terms “(1-6C)alkyl” and “(1-4C)alkyl”. Typical (1-6C)alkyl groups include methyl, ethyl, n-propyl, isopropyl, n-butyl, isobutyl, sec-butyl, t-butyl, n-pentyl, isopentyl, neopentyl, and n-hexyl.

The term “an amino acyl bonded through the carbonyl to form an amine terminus” means an amino acyl bonded through it's carbonyl to an amine containing molecule to form a new amine terminus.

The term “an amino acyl bonded through the amine to form an carboxy terminus” means an amino acyl bonded through it's amine to a carbonyl containing molecule to form a new carboxy terminus.

The term “amino acyl” means an amino acyl derived from an amino acid selected from the group consisting of natural and unnatural amino acids as defined herein. Preferred amino acids are those possessing an α-amino group. The amino acids may be neutral, positive or negative depending on the substituents in the side chain. “Neutral amino acid” means an amino acid containing uncharged side chain substituents. Exemplary neutral amino acids include alanine, valine, leucine, isoleucine, proline, phenylalanine, tryptophan, methionine, glycine, serine, threonine and cysteine. “Positive amino acid” means an amino acid in which the side chain substituents are positively charged at physiological pH. Exemplary positive amino acids include lysine, arginine and histidine. “Negative amino acid” means an amino acid in which the side chain substituents bear a net negative charge at physiological pH. Exemplary negative amino acids include aspartic acid and glutamic acid. Preferred amino acids are α-amino acids. The most preferred amino acids are α-amino acids having L stereochemistry at the α-carbon. Exemplary natural α-amino acids are L-valine, L-isoleucine, L-proline, L-phenylalanine, L-tryptophan, L-methionine, glycine, L-serine, L-threonine, L-cysteine, L-tyrosine, L-asparagine, L-glutamine, L-lysine, L-arginine, L-histidine, L-aspartic acid and L-glutamic acid. “Unnatural amino acid” means an amino acid for which there is no nucleic acid codon. Examples of unnatural amino acids include, for example, the D-isomers of the natural α-amino acids as indicated above; Aib (aminobutyric acid), βAib (3-aminoisobutyric acid), Nva (norvaline), β-Ala, Aad (2-aminoadipic acid), βAad (3-aminoadipic acid), Abu (2-aminobutyric acid), Gaba (γ-aminobutyric acid), Acp (6-aminocaproic acid), Dbu (2,4-diaminobutryic acid), α-aminopimelic acid, TMSA (trimethylsilyl-Ala), aIle (allo-isoleucine), Nle (norleucine), tert-Leu, Cit (citrulline), Orn, Dpm (2,2′-diaminopimelic acid), Dpr (2,3-diaminopropionic acid), α-orβ-Nal, Cha (cyclohexyl-Ala), hydroxyproline, Sar (sarcosine), O-methyl tyrosine, phenyl glycine and the like; cyclic amino acids; N^{a}-alkylated amino acids where N^{a}-alkylated amino acid is N^{a}-(1-10C)alkyl amino acid such as MeGly (N^{a}-methylglycine), EtGly (N^{a}-ethylglycine) and EtAsn (N^{a}-ethylasparagine); and amino acids in which the α-carbon bears two side-chain substituents. Preferred unnatural α-amino acids are D-phenylglycine and L-phenylglycine. The names of natural and unnatural amino acids and residues thereof used herein follow the naming conventions suggested by the IUPAC-IUB Joint Commission on Biochemical Nomenclature (JCBN) as set out in “Nomenclature and Symbolism for Amino Acids and Peptides (Recommendations, 1983)” European Journal of Biochemistry, 138, 9-37 (1984). To the extent that the names and abbreviations of amino acids and residues thereof employed in this specification and appended claims differ from those noted, differing names and abbreviations will be made clear.

The term “(2-4C)alkenyl” represents straight or branched unsaturated alkyl chains having from two to four carbon atoms, and having one or more carbon-carbon double bond, such as, dienes. This group also includes both E and Z isomers. Representative radicals for this group include vinyl, allyl, allenyl, 1-butenyl, 2-butenyl, 2-methyl-1-propenyl, 3-butenyl, 2-methyl-2-propenyl, butadienyl.

The term “(2-4C)alkynyl” means an aliphatic hydrocarbon group containing a carbon-carbon triple bond and which may be straight or branched having about 2 to about 4 carbon atoms in the chain. Preferred alkynyl groups have 2 to about 4 carbon atoms in the chain. Branched means that one or more lower alkyl groups such as methyl or ethyl are attached to a linear alkynyl chain.

The term “aryl” represents groups such as phenyl, substituted phenyl, and naphthyl. The term “arylalkyl” represents a (1-4C)alkyl group bearing one or more aryl groups. Representatives of this latter group include benzyl.

While all the compounds of formula I of the present invention are believed to provide improved oral exposure, certain compounds of the invention are preferred for such use. Preferably, R¹¹ is C(O)YR¹⁴, R¹², R¹³ and A¹ are hydrogen, and X is A². Representative compounds from this preferred group of formula I compounds include (1S,2S,5R,6S)2-amino-2-((1′S) 1-carboxy-3-methylbutylcarbamoyl)-bicyclo[3.1.0]hexane-6-carboxylic acid.

While all the compounds of formula III of the present invention where R^{m} is an amine protecting group are believed to be useful for the synthesis of compounds of formula I, certain compounds are preferred. Preferably, R^{m} is tert-butoxycarbonyl, X and Y are O, R¹³ is hydrogen and R¹⁴ is (1-10C)alkyl, for example a methyl group; or R^{m} is allyloxycarbonyl, X and Y are O, R¹³ is hydrogen and R¹⁴ is (2-10C)alkenyl group, for example an allyl group.

Also useful for the synthesis of compounds of formula I are compounds where the C-2 amino and carboxy groups of the cyclopentane ring are protected in the form of a cyclized ring. Preferably, the cyclized ring is an oxazolidinone that is spiro fused to the 2-postion of bicyclo[3.1.0]hexane-6-carboxylic acid, for example a compound of formula IV.

The compounds of formula I of the present invention are generally synthesized from compounds of formula II where Y is O, and R¹², R¹³ and R¹⁴ are all hydrogen. The compounds of formula II are prepared as described in U.S. Pat. No. 5,750,566 which is incorporated by reference in its entirety.

Generally, compounds of formula I in which X is A² may be prepared by reacting compounds of formula III in which X is O, R¹³ is hydrogen and R^{m} is an amine protecting group. Alternatively, compounds of formula I in which X is A² may be prepared by reacting compounds of formula IV.

More specifically, compounds of formula II are reacted with amine protecting agents such as allyl chloroformate in the presence of a suitable aqueous base such as sodium bicarbonate in a suitable solvent such as dioxane to produce compounds of formula III in which R^{m} is allyloxycarbonyl. Compounds of formula III are then reacted with carboxy protecting agents such as allyl alcohol, EDCI and HOBt in the presence of a suitable base such as triethylamine in a convenient solvent such as dichloromethane to provide compounds of formula V as shown in scheme 2.

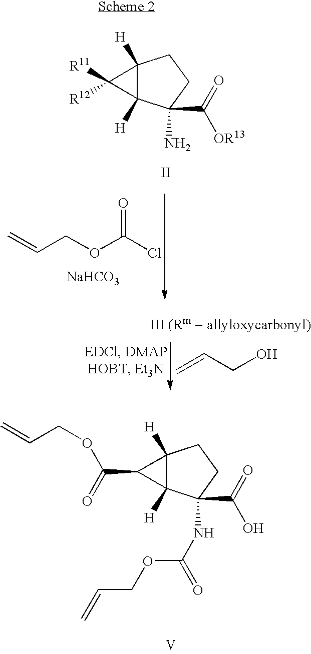

Compounds of formula V are reacted with a carbodiimide such as EDCI and an amino acyl of formula HA²R¹³ in the presence of a suitable base such as triethyl amine to provide compounds of formula VI in which X is A² as shown in scheme 3. The reaction is conveniently performed in the presence of activating agents such as hydroxybenzotriazole and dimethylaminopyridine. Convient solvents include dichloromethane.

Compounds of formula VI in which X is A² are reacted with a metal catalyst such as tetrakistriphenyl phosphine palladium(O) to produce compounds of formula I in which X is A² and Y is O. The reaction is performed in the presence of a metal catalyst regenerating agent such as 1,3-dimethylbarbituric acid in a convenient solvent such as dichloromethane. The acid addition salts may be prepared by the reaction of an acid such as hydrogen chloride gas with a compound of formula I. Convenient solvents include ethyl acetate.

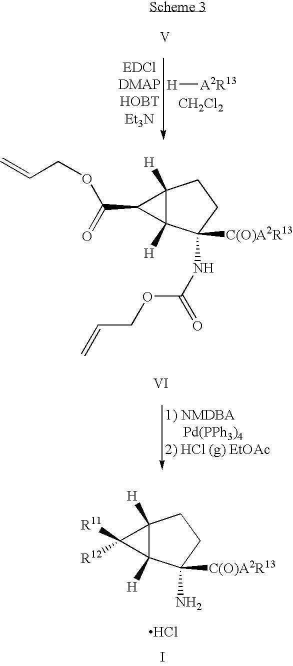

Compounds of formula I in which X is A² may also be prepared from compounds of formula III in which R^{m} is an amine-protecting group such as tert-butyl-oxycarbonyl. More specificly, compounds of formula II are reacted with carboxy protecting agents such as hydrogen chloride gas in methanol to provide a compound of formula VII, as shown in scheme 4.

Compounds of formula VII are reacted with amine protecting agents such as di-tert-butyl dicarbonate in the presence of a suitable base such as potassium carbonate to provide compounds of formula of VIII. Convient solvents include mixtures of dioxane and water.

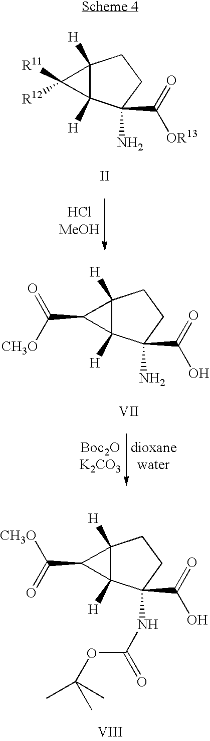

Compounds of formula VIII are reacted with a carbodiimide such as EDCI and an amino acyl of formula HA²R¹³ in the presence of a suitable base such as triethyl amine to provide compounds of formula IX in which X is A², as shown in scheme 5. The reaction is conveniently performed in the presence of activating agents such as hydroxybenzotriazole and dimethylaminopyridine. Convient solvents include dichloromethane.

Compounds of formula IX are reacted with carboxy deprotecting agents such as aqueous lithium hydroxide in a suitable solvent such as tetrahydrofuran and amine deprotecting agents such as hydrogen chloride gas in a suitable solvent such as ethyl acetate to provide compounds of formula I in which X is A².

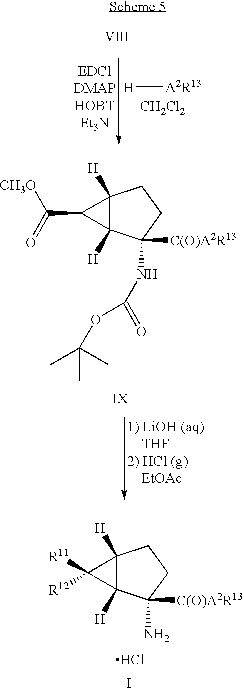

Alternativly, compounds of formula I in which X is A² may be prepared by reacting compounds of formula IV in which Y is O and R¹⁴ is hydrogen as shown in scheme 6. More specifically, compounds of formula IV may be prepared by reacting compounds of formula III in which R^{m} is an amine protecting group such as allyloxycarbonyl with an aldehyde such as paraformaldehyde in the presence of a suitable acid catalyst such as para-toluenesulphonic acid. The reaction may be carried out in a suitable solvent such as benzene with convenient removal of water such as azetropic distillation.

Compounds of formula IV are reacted with an amino acyl of formula HA²R¹³ to provide compounds of formula X in which X is A². Convenient solvents include toluene.

Compounds of formula X are reacted with a metal catalyst such as tetrakistriphenyl phosphine palladium(O) to produce compounds of formula I in which X is A² and Y is O. The reaction is performed in the presence of a metal catalyst regenerating agent such as 1,3-dimethylbarbituric acid in a convenient solvent such as dichloromethane. The acid addition salts may be prepared by the reaction of an acid such as hydrogen chloride gas with a compound of formula I. Convenient solvents include ethyl acetate.

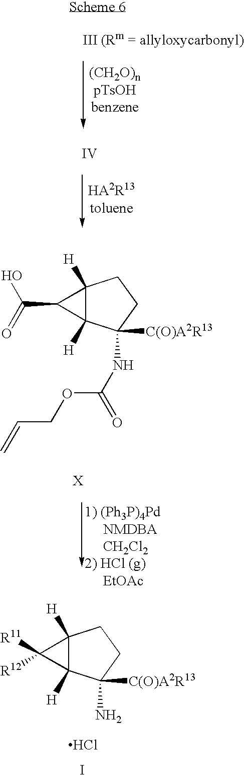

Generally, compounds of formula I in which Y is A² may be prepared by reacting compounds of formula IV in which Y is O and R¹⁴ is hydrogen. More specifically, compounds of formula IV are reacted with a carbodiimide such as EDCI and an amino acyl of formula HA²R¹⁴ in the presence of a suitable base such as triethyl amine to provide compounds of formula XI in which Y is A², as shown in scheme 7. The reaction is conveniently performed in the presence of activating agents such as hydroxybenzotriazole and dimethylaminopyridine. Convenient solvents include dichloromethane.

Compounds of formula XI are reacted with a metal catalyst such as tetrakistriphenyl phosphine palladium(O) to produce compounds of formula I in which Y is A² and X is O. The reaction is performed in the presence of a metal catalyst regenerating agent such as 1,3-dimethylbarbituric acid in a convenient solvent such as dichloromethane. The acid addition salts may be prepared by the reaction of an acid such as hydrogen chloride gas with a compound of formula I. Convenient solvents include ethyl acetate.

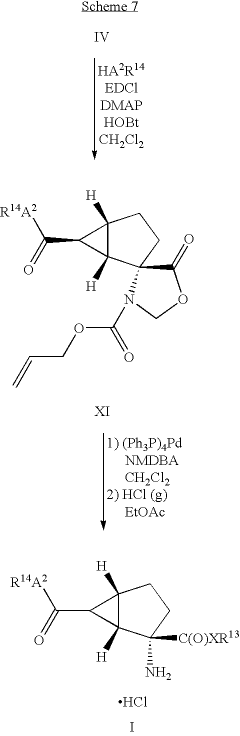

The term “affecting” refers to a formula I compound acting as an agonist at an excitatory amino acid receptor. The term “excitatory amino acid receptor” refers to a metabotropic glutamate receptor, a receptor that is coupled to cellular effectors via GTP-binding proteins. The term “cAMP-linked metabotropic glutamate receptor” refers to a metabotropic receptor that is coupled to inhibition of adenylate cyclase activity.

The term “neurological disorder” refers to both acute and chronic neurodegenerative conditions, including cerebral deficits subsequent to cardiac. bypass surgery and grafting, cerebral ischemia (for example stroke resulting from cardiac arrest), spinal cord trauma, head trauma, Alzheimer's Disease, Huntington's Chorea, amyotrophic lateral sclerosis, AIDS-induced dementia, perinatal hypoxia, hypoglycemic neuronal damage, ocular damage and retinopathy, cognitive disorders, idiopathic and drug-induced Parkinson's Disease. This term also includes other neurological conditions that are caused by glutamate dysfunction, including muscular spasms, migraine headaches, urinary incontinence, drug tolerance, withdrawal, and cessation (i.e. opiates, benzodiazepines, nicotine, cocaine, or ethanol), smoking cessation, emesis, brain edema, chronic pain, sleep disorders, convulsions, Tourette's syndrome, attention deficit disorder, and tardive dyskinesia.

The term “psychiatric disorder” refers to both acute and chronic psychiatric conditions, including schizophrenia, anxiety and related disorders (e.g. panic attack, stress-related cardiovascular disorders or generalized anxiety disorder (GAD)), depression, bipolar disorders, psychosis, and obsessive compulsive disorders.

Of the psychiatric disorders listed above, schizophrenia, anxiety and related disorders, such as GAD or panic attack, depression, bipolar disorder, psychosis and obsessive compulsive disorders are preferred. Of the neurological disorders above, drug tolerance, withdrawal and cessation are preferred.

Thus, in a preferred embodiment, the present invention provides a method for treating anxiety and related disorders such as panic attack or GAD comprising: administering to a patient in need thereof an effective amount of a compound of Formula I, or a pharmaceutically acceptable salt thereof.

As used herein the term “effective amount” refers to the amount or dose of the compound, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances. For example, a typical daily dose may contain from about 25 mg to about 300 mg of the active ingredient. The compounds can be administered by a variety of routes including oral, rectal, transdermal, subcutaneous, intravenous, intramuscular, bucal or intranasal routes. Alternatively, the compound may be administered by continuous infusion.

As used herein the term “patient” refers to a mammal, such as a mouse, guinea pig, rat, dog or human. It is understood that the preferred patient is a human.

The term “treating” (or “treat”) as used herein includes its generally accepted meaning which encompasses prohibiting, preventing, restraining, and slowing, stopping, or reversing progression of a resultant symptom. As such, the methods of this invention encompass both therapeutic and prophylactic administration.

If not commercially available, the necessary starting materials for the above procedures may be made by procedures which are selected from standard techniques of organic and heterocyclic chemistry, techniques which analogous to the syntheses of known, structurally similar compounds, and the procedures described in the Examples, including novel procedures.

A further aspect of the present invention provides for a method of administering an effective amount of a compound of formula II, where R¹³ and R¹⁴ are both hydrogen (a di-acid), which comprises administering to a patient requiring modulated excitatory amino acid neurotransmission a pharmaceutically-effective amount of a compound of formula I.

The ability of compounds to modulate metabotropic glutamate receptor function may be demonstrated by examining their ability to influence either cAMP production (mGluR 2, 3, 4, 6, 7 or 8) or phosphoinositide hydrolysis (mGluR 1 or 5) in cells expressing these individual human metabotropic glutamate receptor (mGluR) subtypes. (D. D. Schoepp, et al., Neuropharmacol., 1996, 35, 1661-1672 and 1997, 36, 1-11).

The ability of formula I compounds to treat anxiety or a related disorder may be demonstrated using the well known fear potentiated startle and elevated plus maze models of anxiety described respectively in Davis, Psychopharmacology, 62:1;1979 and Lister, Psychopharmacol, 92:180-185; 1987

### In Vivo Exposure as Measured by Rat Plasma Concentration

To study the in vivo exposure of LY354740 following oral dosing of compounds of the present invention in comparison to LY354740, studies measuring the plasma concentrations of LY354740 in rats were performed.

Mature Fischer 344 male rats (190-270 gram) were obtained from Harlan Sprague-Dawley, Cumberland, Ind., USA and acclimated in the study housing for 3 days. On day 4, test compounds were dissolved in buffered water (1 mg/ml=test compound/20 mM potassium dihydrogen phosphate, pH=2) and given orally as a single 5 mg/kg dose. Blood samples were collected through orbital sinus or cardiac puncture (last time point) at 0.5 and 1 hour or, alternatively, 1 and 3 hours. Plasma samples were stored at −20° C. in the presence of phenylmethylsulfonyl fluoride, a protease inhibitor, prior to analysis. Plasma samples and internal standard compounds were pretreated by solid phase extraction (SAX support, methanol/water/dilute acetic acid). The plasma concentrations (ng/ml) of LY354740 for each test compound were determined by LC/MS/MS and are presented as a sum of the concentrations at the 0.5 and 1 hour or, alternatively, 1 and 3 hour sample time points as shown in table 1.

TABLE 1
Comparison of plasma concentrations of LY354740 and
compounds of the present invention

| | Plasma Concentration of |
|---|---|
| Compound | LY354740, ng/ml |
| (@5 mg/kg p. o.) | (sum of 0.5 and 1 hour) |
| LY354740 | 466 |
| Example 1 | 1248 |

The compounds of the present invention are preferably formulated prior to administration. Therefore, another aspect of the present invention is a pharmaceutical formulation comprising a compound of formula I a pharmaceutically acceptable metabolically labile ester thereof, or a pharmaceutically acceptable salt thereof, and a pharmaceutically-acceptable carrier, diluent, or excipient. The present pharmaceutical formulations are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragacanth, gelatin, calcium silicate, microcrystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 5 mg to about 500 mg, more preferably about 25 mg to about 300 mg of the active ingredient. As used herein, the term “active ingredient” refers to a compound included within the scope of formula I.

The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient.

The Examples are not intended to be limiting to the scope of the invention in any respect, and should not be so construed. All experiments were run under a positive pressure of dry nitrogen or argon. All solvents and reagents were purchased from commercial sources and used as received, unless otherwise indicated. Dry tetrahydrofuran (THF) was obtained by distillation from sodium or sodium benzophenone ketyl prior to use. Proton nuclear magnetic resonance (¹H NMR) spectra were obtained on a GE QE-300 spectrometer at 300.15 MHz, a Bruker AM-500 spectrometer at 500 MHz, a Bruker AC-200P spectrometer at 200 MHz or a Varian Inova at 500 MHz. Free atom bombardment mass spectroscopy (FABMS) was performed on a VG ZAB-2SE instrument. Field desorption mass spectroscopy (FDMS) was performed using either a VG 70SE or a Varian MAT 731 instrument. Optical rotations were measured with a Perkin-Elmer 241 polarimeter. Chromatographic separation on a Waters Prep 500 LC was generally carried out using a linear gradient of the solvents indicated in the text. The reactions were generally monitored for completion using thin layer chromatography (TLC). Thin layer chromatography was performed using E. Merck Kieselgel 60 F254 plates, 5 cm×10 cm, 0.25 mm thickness. Spots were detected using a combination of UV and chemical detection (plates dipped in a ceric ammonium molybdate solution [75 g of ammonium molybdate and 4 g of cerium (IV) sulfate in 500 mL of 10% aqueous sulfuric acid] and then heated on a hot plate). Flash or silica gel chromatography was performed as described by Still, et al. Still, Kahn, and Mitra, J. Org. Chem., 43, 2923 (1978). Elemental analyses for carbon, hydrogen, and nitrogen were determined on a Control Equipment Corporation 440 Elemental Analyzer, or were performed by the Universidad Complutense Analytical Centre (Facultad de Farmacia, Madrid, Spain). Melting points were determined in open glass capillaries on a Gallenkamp hot air bath melting point apparatus or a Büchi melting point apparatus, and are uncorrected. The number in parenthesis after the compound name refers to the compound number.

The abbreviations, symbols and terms used in the examples have the following meanings.

Ac=acetyl

AllocCl=allyl chloroformate

Anal.=elemental analysis

Bn or Bzl=benzyl

Bu=butyl

BOC=tert-butoxycarbonyl

calcd=calculated

D₂O=deuterium oxide

DCC=dicyclohexylcarbodiimide

DIBAL-H=diisobutyl aluminum hydride

DMAP=dimethylaminopyridine

DMF=dimethylformamide

DMSO=dimethylsulfoxide

EDCI=N-ethyl-N′N′-dimethylaminopropyl

carbodiimide

Et=ethyl

EtOAc=ethyl acetate

EtOH=ethanol

FAB=Fast Atom Bombardment (Mass Spectrascopy)

FDMS=field desorption mass spectrum

HOAt=1-hydroxy-7-azabenzotriazole

HOBt=1-hydroxybenzotriazole

HPLC=High Performance Liquid Chromatography

HRMS=high resolution mass spectrum

i-PrOH=isopropanol

IR=Infrared Spectrum

L=liter

Me=methyl

MeOH=methanol

MPLC=Medium Pressure Liquid Chromatography

Mp=melting point

MTBE=t-butyl methyl ether

NBS=N-bromosuccinimide

NMDBA=1,3-dimethylbarbituric acid

NMR=Nuclear Magnetic Resonance

p-TsOH=para-toulene sulphonic acid

Ph=phenyl

p.o.=oral administration

i-Pr=isopropyl

Rochelle's Salt=potassium sodium tartrate

SM=starting material

TBS=tert-butyldimethylsilyl

TEA=triethylamine

Temp. =temperature

TFA=trifluoroacetic acid

THF=tetrahydrofuran

TLC=thin layer chromatography

t-BOC=tert-butoxycarbonyl

### EXAMPLE PREPARATIONS 1-3

### Describe Synthesis Methods for Intermediates

### EXAMPLE PREPARATION 1

### Synthesis of (1S,2S,5R,6S)-2-allyloxycarbonylamino-bicyclo[3.1.0]hexane-2,6-dicarboxylic acid 6-allyl ester

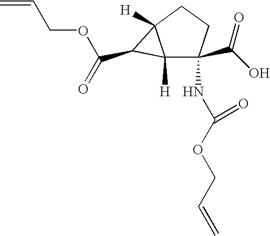

### A. (1S,2S,5R,6S)-2-Allyloxycarbonylamino-bicyclo[3.1.0]hexane-2,6-dicarboxylic acid

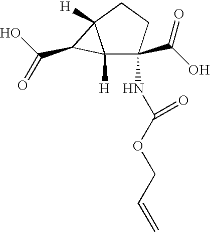

(1S,2S,5R,6S)-2-Amino-bicyclo[3.1.0]hexane-2,6-dicarboxylic acid (15.0 g, 73.9 mmol) was slowly dissolved in 250 mL of NaHCO₃ sat. (250 mL). After complete solution, dioxane (100 mL) and allyl chloroformate (15.7 mL, 147.8 mmol) were added at room temperature and the mixture was stirred overnight. The reaction mixture was diluted with water (100 mL) and washed with ethyl acetate (3×). The organic layer was extracted once with sat. NaHCO₃. The combined aqueous layers were acidified to pH 1 with 4N HCl and extracted with ethyl acetate (2×). The organic layer was dried over magnesium sulfate, filtered and concentrated to provide an oil (13.4 g, 67% yield) that was used without further purification.

¹H-NMR (CD₃OD)δ: 6.01-5.82 (m, 1 H); 5.35-5.13 (m, 2 H); 4.51 (d, J=5.1 Hz, 2 H); 2.48-1.78 (m, 5 H); 1.69-1.62 (m, 1 H); 1.45-1.29 (m, 1 H). ¹³C-NMR (CD₃OD)δ: 176.7, 176.6, 158.3, 134.2, 117.4, 67.3, 66.3, 35.8, 33.1, 29.9, 27.0, 22.0.

### B. (1S,2S,5R,6S)-2-Allyloxycarbonylamino-bicyclo[3.1.0]hexane-2, 6-dicarboxylic acid 6-allyl ester

To a suspension of (1S,2S,5R,6S)-2-allyloxycarbonylamino-bicyclo[3.1.0]hexane-2,6-dicarboxylic acid (13.4 g, 49.8 mmol) in dichloromethane (400 mL), N-ethyl-N′-dimethylaminopropylcarbodiimide (9.55 g, 49.8 mmol) and dimethylaminopyridine (0.61 g, 5.0 mmol) were added at room temperature under nitrogen. Allyl alcohol (3.4 mL, 49.8 mmol) was added and the mixture was stirred overnight at room temperature. The reaction mixture was diluted with dichloromethane and washed with water (2×). The organic layer was dried over magnesium sulfate, filtered and concentrated to provide the title compound (6.8 g, 44% yield) as an oil.

¹H-NMR (CD₃ OD)δ: 6.01-5.82 (m, 1 H); 5.35-5.13 (m, 2 H); 4.51 (d, J=5.1 Hz, 2 H); 2.48-1.78 (m, 5 H); 1.69-1.62 (m, 1 H); 1.45-1.29 (m, 1 H).

### EXAMPLE PREPARATION 2

### Synthesis of (1S,2S,5R,6S)-2-tert-butoxycarbonylamino-bicyclo[3.1.0]hexane-2,6-dicarboxylic acid 6-methyl ester

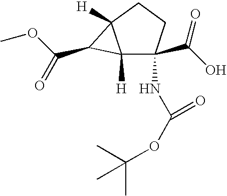

### A. (1S,2S,5R,6S)-2-Amino-bicyclo[3.1.0]hexane-2,6-dicarboxylic acid 6-methyl ester hydrochloride

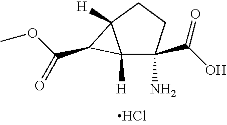

A 250 ml flask was charged with (1S,2S,5R,6S)-2-amino-bicyclo[3.1.0]hexane-2,6-dicarboxylic acid monohydrate (18.08 g, 0.089 mol) and 90 ml of a 2.5 M HCl gas solution in methanol was added. The suspension was vigorously stirred and methanol (50 ml) was added after 10 min. The solution was stirred for 5 hours. Diethyl ether was added and the solid was filtered and rinsed with diethyl ether. After drying under high vacuum a fine powdered solid was obtained. (15.5 g, 87% yield)

mp 245° C. (dec.) [α]_{D}²⁵=+32.2° (c=1.25, MeOH). ¹H NMR (D₂O) δ: 3.65 (s, 3H), 2.30-2.00 (m, 6H), 1.56 (m, 1H). ¹³C NMR (D₂O) δ: 174.4, 172.9, 65.9, 52.6, 32.6, 30.0, 29.2, 26.2, 21.2.

### B. (1S,2S,5R,6S)-2-tert-Butoxycarbonylamino-bicyclo[3.1.0]hexane-2,6-dicarboxylic acid 6-methyl ester

(1S,2S,5R,6S)-2-Amino-bicyclo[3.1.0]hexane-2,6-dicarboxylic acid 6-methyl ester (8.6 g, 43.2 mmol), di-tert-butyl dicarbonate (18.8 g, 86.3 mmol) and potassium carbonate (11.9 g, 86.3 mmol) were dissolved in dioxane (200 mL) and water (100 mL). The solution was stirred at room temperature for 2 days. Hydrochloric acid (1N) was added dropwise to pH 1, and it was extracted with ethyl acetate. The organic layer was washed with brine, dried over MgSO₄, filtered and concentrated to provide the title compound as a white solid (9.7 g, 75% yield)

mp 164-165° C. [α]_{D}²⁵=−30.1° (c=1.6, MeOH). ¹H NMR (CDCl₃) δ: 3.65 (s, 3 H); 2.45-1.87 (m, 5 H); 1.71 (t, J=2.9 Hz, 1 H); 1.41 (s, 9 H); 1.30-1.09 (m, 1 H). ¹³C NMR (CDCl₃) δ: 177.8, 172.9, 156.2, 81.6, 66.4, 51.7, 34.7, 32.5, 28.4, 28.1 (3C), 26.4, 21.0.

### EXAMPLE PREPARATION 3

### Synthesis of (1S,2S,5R,6S)-3-allyloxycarbonyl-5-oxo-oxazolidine-4-spiro-2′-bicyclo[3,1,0]-hexane-6′-carboxylic acid

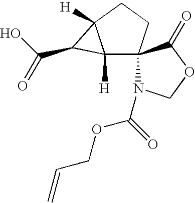

(1S,2S,5R,6S)-2-Allyloxycarbonylamino-bicyclo[3.1.0]hexane-2,6-dicarboxylic acid (19.2 g, 64.3 mmol), para-formaldehyde (7.7 g, 257.3 mmol) and p-toluenesulphonic acid (0.61 g, 3.2 mmol) were refluxed in 200 mL of benzene with azeotropic removal of water for 2h. The mixture was cooled to room temperature, diluted with 200 mL of ethyl acetate, washed with brine, dried over MgSO₄, filtered and concentrated to afford a slighly hygroscopic solid. (17.6 g, 97%) ¹H-NMR (CDCl₃)δ: 6.10-5.90 (m, 1 H); 5.41-5.21 (m, 4 H); 4.65 (dt, J=5.6, 1.3 Hz, 2 H); 2.51-2.49 (m, 1 H); 2.33-2.18 (m, 2 H); 2.04-1.92 (m, 3 H); 1.75-1.70 (m, 1 H). ¹³C-NMR (CD₃OD): 176.1, 175.4, 153.0, 133.5, 118.9, 78.4, 67.8, 67.4, 33.1, 27.2 (×2), 26.0, 23.9.

### General Procedures 1-9

### Describe General Synthesis Procedures

### General Procedure 1

### General Procedure for the Coupling of (1S,2S,5R,6S)-2-allyloxycarbonylamino-bicyclo[3.1.0]hexane-2,6-dicarboxylic acid 6-allyl ester with amines.

For 2 mmol scale: To a suspension of (1S,2S,5R,6S)-2-Allyloxycarbonylamino-bicyclo[3.1.0]hexane-2,6-dicarboxylic acid 6-allyl ester (1.0 equiv.), N-ethyl-N′-dimethylaminopropylcarbodiimide (1.4 equiv), dimethylaminopyridine (0.1 equiv), N-hydroxybenzotriazole (1.2 equiv) and the amine (1.2 equiv) in dichloromethane under nitrogen was added triethylamine (2 equiv). The resulting solution was stirred overnight at room temperature or until the TLC analysis showed the absence of starting material. The solution was concentrated under vacuum, slurried in ethyl acetate (50 mL) and washed twice with 1N HCl. The organic layer was dried over MgSO₄, concentrated and purified by column chromatography (silica gel, hexanes/ethyl acetate, 3/1).

### General Procedure 2

### General Procedure for the Coupling of (1S,2S,5R,6S)-2-allyloxycarbonylamino-bicyclo[3.1.0]hexane-2,6-dicarboxylic acid 6-allyl ester with amines

For 2 mmol scale: The amine (1.3 equiv), dimethylaminopyridine (0.1 equiv), a slurry of 5-([1,4′]bipiperidinyl-1′-sulfonyl)-benzotriazol-1-ol in 20 mL of dimethylformamide previously heated at 602° C. (1.2 equiv), a solution of N-ethyl-N′-dimethylaminopropylcarbodiimide (1.4 equiv) and (1S,2S,5R,6S)-2-allyloxycarbonylamino-bicyclo[3.1.0]hexane-2,6-dicarboxylic acid 6-allyl ester (1.0 equiv) in 20 mL of dichloromethane and triethylamine (1.2 equiv) were mixed in a screw capped tube and stirred in an orbital agitator overnight. Dichloromethane was removed under vacuum, the DMF solution was diluted with 150 mL of ethyl acetate and washed 1N HCl and brine. The organic layer was dried over MgSO₄, filtered and concentrated to provide the product.

### General Procedure 3

### General Procedure for the Coupling of (1S,2S,5R,6S)-2-tert-butoxycarbonylamino-bicyclo[3.1.0]hexane-2,6-dicarboxylic acid 6-methyl ester with amines

The same procedure as General Procedure 1 but using (1S,2S,5R,6S)-2-tert-butoxycarbonylamino-bicyclo[3.1.0]hexane-2,6-dicarboxylic acid 6-methyl ester as starting material.

### General Procedure 4

### General Procedure for the Ring-Opening of (1S,2S,5R,6S)-3-allyloxycarbonyl-5-oxo-oxazolidine-4-spiro-2′-bicyclo[3,1,0]-hexane-6′-carboxylic acid with amines

(1S,2S,5R,6S)-3-Allyloxycarbonyl-5-oxo-oxazolidine-4-spiro-2′-bicyclo[3,1,0]-hexane-6′-carboxylic acid (1.0 equiv) was suspended in the appropriate solvent (0.17 M solution) and heated at 90° C. to homogeneity. The corresponding amine (2.0 equiv) was added and the mixture was refluxed overnight. The reaction mixture was cooled and dissolved in ethyl acetate. The organic layer was washed with 1N hydrochloric acid (2×) and brine, dried over magnesium sulfate and concentrated to dryness. The resulting residue was purified by silica gel chromatography.

### General Procedure 5

### General Procedure for the Coupling of (1S,2S,5R,6S)-3-allyloxycarbonyl-5-oxo-oxazolidine-4-spiro-2′-bicyclo[3,1,0]-hexane-6′-carboxylic acid with Aminoesters

Same as General Procedure 1 but using (1S,2S,5R,6S)-3-allyloxycarbonyl-5-oxo-oxazolidine-4-spiro-2′-bicyclo[3,1,0]-hexane-6′-carboxylic acid as starting material.

### General Procedure 6

### General Procedure for 2-allyloxycarbonylamino Deprotection: Hydrochloride Formation

The corresponding allylated compound (1.0 equiv) was dissolved in dry dichloromethane (0.1 M solution) under nitrogen. 1,3-Dimethylbarbituric acid (3.0 equiv for each allyl group to be removed) and tetrakis(triphenylphosphine)-palladium (0) (0.03 equiv) were added and the solution was heated at 35° C. for 2 h. After cooling to room temperature, the solvent was removed under vacuum and the resulting residue was dissolved in a solution of ethyl acetate saturated with hydrogen chloride gas and stirred for 2 h. In the case where a solid appeared, the reaction was filtered and the filtrate was washed with ethyl acetate and ether. In the case where a solid did not appear, the solvent was removed under vacuum and the residue was stirred with ether overnight. The solid was filtered, washed thoroughly with ether and dried to provide the product.

### General Procedure 7

### General Procedure for 2-allyloxycarbonylamino Deprotection: SCX Purification

(For 1 mmol scale): The allyl derivative (1 equiv), dimethylbarbituric acid (4 equivalents), tetrakis triphenylphosphine palladium (0) (0.03 equivalents) and dichloromethane (5 mL) were stirred at 30° C. overnight. The reaction was diluted with 1.5 mL of dimethylformamide and the resulting solution was filtered through a 2 g Bond-Elut SCX (cation exchange) cartridge. The cartridge was washed with 50 mL of dichloromethane and eluted with 15 mL of 2N ammonia in methanol. The eluted fractions were evaporated to provide the product.

### General Procedure 8

### General Procedure for 2-allyloxycarbonylamino Deprotection: Zwitterion Formation

The corresponding allylated compound (1.0 equiv) was dissolved in dry dichloromethane (0.1 M solution) under nitrogen. 1,3-Dimethylbarbituric acid (0.5 equiv for each allyl group to be removed) and tetrakis(triphenylphosphine)-palladium (0) (0.02 equiv) were added and the resulting solution was heated at 35° C. for 2 h. After cooling at room temperature, if a solid was formed, it was filtered and washed with dichloromethane, ethyl acetate, diethyl ether and dried to provide the product. In case the solid did not appear, the solvent was removed under vacuum and a large amount of ethyl ether was added. After stirring for 30 min the solid was filtered and washed with ether, ethyl acetate and dried to provide the product.

### General Procedure 9

### General Procedure for the Deprotection of (1S,2S,5R,6S)-2-tert-butoxycarbonylamino-bicyclo[3.1.0]hexane-2,6-dicarboxylic acid 6-methyl ester derivatives

The corresponding 2-N-BOC-6-methyl ester dipeptide (1.0 equiv) was dissolved in THF and an equal volume of 2.5 N aqueous LiOH (10-20 equiv) was added. The reaction mixture was stirred at room temperature for 1-3 h. After dilution with water, the mixture was washed with ethyl acetate. The aqueous layer was acidified to pH 2 with 1N HCl and extracted with ethyl acetate. The combined-organic extracts were washed with brine, dried over sodium sulfate, filtered and concentrated. The foamy solid was dissolved in a solution of ethyl acetate saturated with hydrogen chloride gas (5-10 ml/mmol) and the resulting mixture was stirred overnight. The solid was filtered, rinsed with diethyl ether and dried under high vacuum to provide the product.

### EXAMPLE 1

### Synthesis of (1S,2S,5R,6S) 2-amino-2-[(1′S)-carboxy-3′-methylbutyl]carbamoyl-bicyclo[3.1.0]hexane-6-carboxylic acid

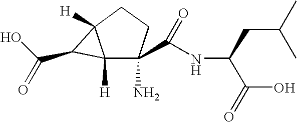

### A. (1S,2S,5R,6S) 2-Allyloxycarbonylamino-2-[(1S)-allyloxycarbonyl-3-methylbutyllcarbamoyl-bicyclo[3.1.0]hexane-6-carboxylic acid allyl ester

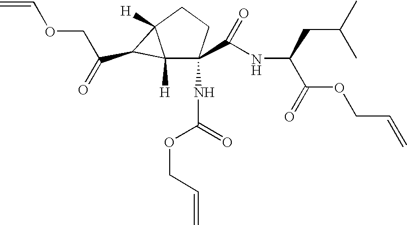

The title compound was prepared from (L)-leucine allyl ester hydrochloride as described in General Procedure 2. (98% yield)

Oil. ¹H-NMR (CDCl₃)δ: 7.13 (br s, 1 H); 5.95-5.79 (m, 2 H); 5.60 (s, 1 H); 5.34-5.07 (m, 7 H); 4.63-4.50 (m, 6 H); 2.49-2.34 (m, 3 H); 2.10-1.85 (m, 4 H); 1.71 (t, J=2.7 Hz, 1 H); 1.41 (d, J=7.3 Hz, 3 H); 1.28-1.12 (m, 1 H).

### B. (1S,2S,5R,6S) 2-Amino-2-[(1′S)-carboxy-3′-methyl-butyl]carbamoyl-bicyclo[3.1.0]hexane-6-carboxylic acid

The title compound was prepared from 2-allyloxycarbonylamino-2-(1′S-allyloxycarbonyl-3′-methyl-butylcarbamoyl)-bicyclo[3.1.0]hexane-6-carboxylic acid allyl ester as described in General Procedure 6. (5.0% yield)

mp 130.5-131.7° C. [α]_{D}²⁵=−53 (c=0.107, H₂O). ¹H-NMR (DMSO-D₆/TFA-D)δ: 4.45-4.38 (m, 1 H); 2.27-2.21 (m, 2 H); 2.11-1.90 (m, 4 H); 1.77-1.54 (m, 4 H); 0.95 (dd, J=6.18, 5.10 Hz, 6 H). ¹³C-NMR (DMSO-D₆/TFA-D)δ: 173.7, 173.2, 170.1, 65.9, 51.2, 31.7, 31.1, 29.5, 25.7, 24.9, 23.4, 21.6, 21.2. MS (Electrospray): 299.16 (M⁺ +H).

### EXAMPLE 2

### Synthesis of (1S,1′S,2S,5R,6S)-2-amino-2-(carboxymethyl-carbamoyl)-bicyclo[3.1.0]hexane-6-carboxylic acid

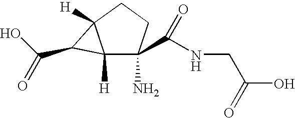

### A. (1S,1′S,2S,5R,6S)-2-Allyloxycarbonylamino-2-(ethoxycarbonylmethyl-carbamoyl)-bicyclo[3.1.0]hexane-6-carboxylic acid allyl ester

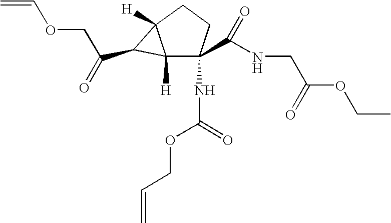

The title compound was prepared from glycine ethyl ester as described in General Procedure 1. (76% yield)

White solid. ¹H-NMR (CDCl₃)δ: 7.02 (m, 1 H); 5.99-5.94 (m, 2 H); 5.64 (bs, 1 H); 5.34-5.16 (m, 4 H); 4.54 (dt, J=5.6, 1.1 Hz, 4 H); 4.19 (q, J=7.3 Hz, 2 H); 4.07-3.99 (m, 2H); 2.52 (b, J=3.5 Hz, 1 H); 2.42 (dd, J=13.2, 8.3 Hz, 1 H); 2.21-2.07 (m, 2 H); 1.94 (dd, J=12.9, 7.8 Hz, 1 H); 1.74 (t, J=3.0 Hz, 1 H); 1.27 (t, J=7.0 Hz, 3 H), 1.30-1.15 (m, 1H).

### B. (1S,1′S,2S,5R,6S)-2-Amino-2-(carboxymethyl-carbamoyl)-bicyclo[3.1.0]hexane-6-carboxylic acid

The title compound was prepared from (1S,1′S,2S,5R,6S)-2-allyloxycarbonylamino-2-(ethoxycarbonylmethyl-carbamoyl)-bicyclo[3.1.0]hexane-6-carboxylic acid allyl ester as described in General Procedure 8. The residue was suspended in 2 mL of tetrahydrofuran and 4 mL of 2.5 N aqueous lithium hydroxide were added. The reaction was stirred for 2 h, acidified to pH 3 with 6N HCl and concentrated to dryness. Purification by ion exchange chromatography provided the title compound. (70% yield)

White solid. ¹H-NMR (D₂O+Pyridine-d₅)δ: 3.75 (s, 2 H); 2.10-2.00 (m, 5 H); 1.76 (t, J=2.7 Hz, 1 H); 1.68-1.55 (m, 1 H). ¹³C-NMR (D₂O+Pyridine-d₅)δ: 179.0, 175.9, 171.2, 66.5, 43.6, 31.1, 29.5, 27.9, 25.0, 24.3.

### EXAMPLE 3

### Synthesis of (1S,2S,5R,6S)-2-amino-2-[(1′R)-carboxy-phenylmethyl]carbamoyl-bicyclo[3.1.0]hexane-6-carboxylic acid

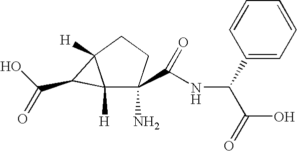

### A. (1S,2S,5R,6S)-2-Allyloxycarbonylamino-2-[(1R)-methoxycarbonylphenylmethyl]carbamoyl-bicyclo[3.1.0]hexane-6-carboxylic acid allyl ester

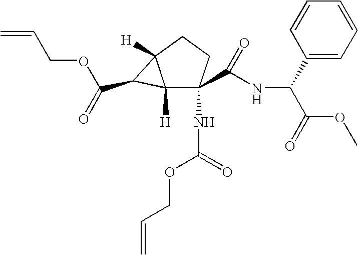

The title compound was prepared from (R)-phenyl glycine methyl ester as described in General Procedure 1. (74% yield)

White solid. ¹H-NMR (CDCl₃)δ: 7.34-7.33 (m, 5 H); 5.98-5.81 (m, 2 H); 5.55 (d, J=7.3 Hz, 1 H); 5.37-5.21 (m, 5 H); 4.58-4.50 (m, 4 H); 3.73 (s, 3 H); 2.53 (bs, 1H); 2.44 (dd, J=13.2, 8.6 Hz, 1 H); 2.29-1.90 (m, 3 H); 1.76 (t, J=3.0 Hz, 1 H); 1.28-1.13 (m, 1 H).

### B. (1S,2S,5R,6S)-2-Amino-2-[(1′R)-carboxyphenyl-methyl]carbamoyl-bicyclo[3.1.0]hexane-6-carboxylic acid

The title compound was prepared from (1S,2S,5R,6S)-2-allyloxycarbonylamino-2-[(1′R)-methoxycarbonyl-phenylmethyl]carbamoyl-bicyclo[3.1.0]hexane-6-carboxylic acid allyl ester as described in General Procedure 8. Half of the residue was suspended in 2 mL of tetrahydrofuran and 4 mL of 2.5N lithium hydroxide was added. The reaction was stirred for 2 h, acidified to pH 3 with 6N HCl and evaporated to dryness. Purification by ion exchange chromatography provided the title compound. (40% yield)

White solid. ¹H-NMR (D₂O+Py-d₅)δ: 7.41-7.35 (m, 5 H); 5.16 (s, 1 H); 2.36-2.10 (m, 5 H); 1.97 (t, J=2.7 Hz, 1 H); 1.73-1.55 (m, 1 H). ¹³C-NMR (D₂O+Py−d₅)δ: 177.9, 174.3, 169.0, 137.4, 127.9, 127.0, 125.8, 65.3, 58.6, 30.0, 29.4, 26.4, 24.5, 23.6.

### EXAMPLE 4

### Synthesis of (1S,2S,5R,6S)-2-amino-2-[(1′S)-carboxy-3′-methylthiopropyl]carbamoyl-bicyclo[3.1.0]hexane-6-carboxylic acid

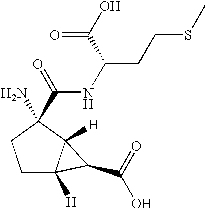

### A. (1S,2S,5R,6S)-2-Allyloxycarbonylamino-2-[(1′S)-methoxycarbonyl-3′-methylthiopropyl]carbamoyl-bicyclo[3.1.0]hexane-6-carboxylic acid allyl ester

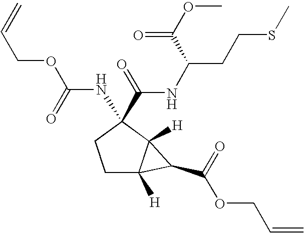

The title compound was prepared from (L)-methionine methyl ester hydrochloride as described in General Procedure 1. (90% yield)

White foamy solid. ¹H-NMR (CDCl₃)δ: 7.21 (brs, 1 H), 5.89 (m, 2 H), 5.40-5.20 (m, 4 H), 4.67 (m, 1 H), 4.52 (d, 4 H, J=7.0 Hz), 3.74 (s, 3 H), 2.55-2.37 (m, 4 H), 2.08 (s, 3 H), 2.30-1.89 (m, 5 H), 1.71 (t, 1 H, J=2.4 Hz), 1.21 (m, 1 H). ¹³C NMR (CDCl₃) δ: 172.5, 172.2, 171.9, 155.7, 132.4, 131.9, 118.3, 117.9, 67.0, 65.9, 65.3, 52.4, 51.5, 34.3, 32.7, 31.2, 28.9, 26.4, 21.2, 15.3.

### B. (1S,2S,5R,6S) 2-Amino-2-[(1′S)-carboxy-3′-methylthiopropyl]carbamoyl-bicyclo[3.1.0]hexane-6-carboxylic acid

The reaction was preformed with (1S,2S,5R,6S)-2-allyloxycarbonylamino-2-[(1′S)-methoxycarbonyl-3′-methylthio propionyl]carbamoyl-bicyclo[3.1.0]hexane-6-carboxylic acid allyl ester as described in General Procedure 8. The solid obtained was dissolved in 10 mL of 2.5N lithium hydroxide and stirred overnight. The solution was acidified to pH 2 with 1 N HCl aqueous layer and extracted with ethyl acetate (4×). The combined organic extracts were dried over MgSO₄ and concentrated to dryness. The resulting material was purified by anion exchange chromatography to provide the title compound. (50% yield)

White solid, mp: 72-74° C. ¹H NMR (D₂O) δ: 4.26 (dd, 1 H, J=8.0, 4.6 Hz), 2.54-2.31 (m, 2 H), 2.00 (s, 3 H), 2.10-1.85 (m, 7 H), 1.77 (t, 1 H, J=2.7 Hz), 1.58 (m, 1 H). ¹³C NMR (D₂O) δ: 178.4, 177.3, 170.6, 66.4, 54.6, 31.2, 30.2, 29.7, 29.5, 28.0, 25.3, 23.9, 14.2. MS (Electrospray): 317 (M⁺+1). [α]_{D}²⁵: −62° (c=0.95, 1N HCl).

### EXAMPLE 5

### Synthesis of (1′S,2′S,5′R,6′S)-2′-amino-6′-carboxy-bicyclo[3.1.0]-hexane-2′-carbonyl-pyrrolidine-2S carboxylic acid

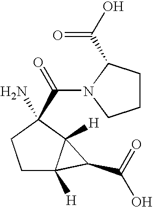

### A. (1S′,2S′,5R′,6S′)-1-(6′-Allyloxycarbonyl-2′-allyloxycarbonylamino-2′-bicyclo[3.1.0]-hexane-2′-carbonyl-pyrrolidine-2S carboxylic acid methyl ester

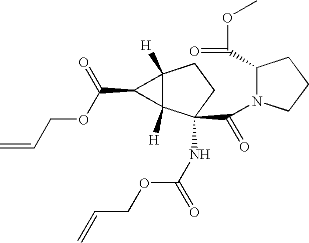

The title compound was prepared from (L)-proline methyl ester hydrochloride as described in General Procedure 1. (69% yield)

White foamy solid. ¹H NMR (CDCl₃) δ: 5.87 (m, 2 H), 5.25 (m, 4 H), 4.54 (d, 4 H, J=5.9 Hz), 3.70 (s, 3 H), 3.69 (m, 1 H), 2.54 (m, 2 H), 2.13-1.85 (m, 7 H), 1.64 (m, 1 H), 1.19 (m, 1 H). ¹³C NMR (CDCl₃) δ: 172.8, 172.2, 170.0, 155.8, 154.7, 132.5, 132.0, 118.0, 117.7, 66.8, 65.6, 65.1, 60.4, 51.9, 47.5, 34.6, 32.5, 31.5, 28.9, 27.8, 25.4, 20.8.

### B. (1′S,2′S,5′R,6′S)-2′-amino-6′-carboxy-bicyclo[3.1.0]-hexane-2′-carbonyl]-pyrrolidine-2S carboxylic acid

The title compound was prepared from (1S′,2S′,5R′,6S′)-1-(6′-allyloxycarbonyl-2′-allyloxycarbonylamino-2′-bicyclo[3.1.0]-hexane-2′-carbonyl]-pyrrolidine-2S carboxylic acid methyl ester as described in General Procedure 8. (89% yield)

White solid, mp: 274 C. ¹H NMR (D₂O) δ: 4.25 (m, 1 H), 2.08 (m, 2 H), 1.82-2.30 (m, 10 H), 1.70 (m, 1 H), 1.44 (m, 1 H). ¹³C NMR (D₂O) δ: 172.1, 169.5, 125.7, 67.1, 58.6, 45.8, 32.7, 32.3, 27.9, 27.1, 25.7, 22.3, 21.8. MS (Electrospray): 303, 265. [α]_{D}²⁵=−103.3° (c=0.92, 1N HCl).

### EXAMPLE 6

### Synthesis of (1S,2S,5R,6S)-2-amino-2-[1′S-carboxy-2′-(4″-hydroxyphenyl)ethyl]carbamoyl-bicyclo[3.1.0]-hexane-6-carboxylic acid

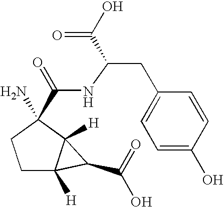

### A. (1S,2S,5R,6S)-2-tert-Butoxycarbonylamino-2-[2-(4-hydroxy-phenyl)-1′S-methoxycarbonylethyl]carbamoyl-bicyclo[3.1.0]-hexane-6-carboxylic acid methyl ester

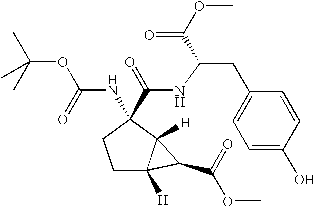

The title compound was prepared from (L)-tyrosine methyl ester hydrochloride as described in General Procedure 3. (95% yield)

White solid. ¹H NMR (CDCl₃) δ: 6.98 (d, 2 H, J=8.2 Hz), 6.73 (d, 2 H, J=8.3 Hz), 5.11 (brs, 1 H), 4.81 (dd, 1 H, J=7.5, 6.1 Hz), 3.71 (s, 3 H), 3.67 (s, 3 H), 3.15 (dd, 1 H, J=13.7, 6.2 Hz), 2.99 (dd, 1 H, J=13.9, 6.2 Hz), 2.45 (dd, 1 H, J=12.6, 7.5 Hz), 2.25-1.82 (m, 4 H), 1.64 (t, 1 H, J=2.9 Hz), 1.40 (s, 9 H), 1.12 (m, 1 H). ¹³C NMR (CDCl₃) δ: 173.0, 172.0, 155.5, 155.5, 155.2, 130.2 (2 C), 127.0, 115.6 (2 C), 80.7, 67.0, 53.4, 52.2, 51.8, 37.1, 35.2, 32.8, 28.7, 28.0 (3 C), 26.8, 21.3.

### B. (1S,2S,5R,6S)-2-Amino-2-[(1′S)-carboxy-2′-(4″-hydroxy-phenyl)ethyl]carbamoyl-bicyclo[3.1.0]-hexane-6-carboxylic acid

The title compound was prepared from (1S,2S,5R,6S)2-tert-butoxycarbonylamino-2-[2′-(4″-hydroxy-phenyl)-1′S-methoxycarbonylethyl]carbamoyl-bicyclo[3.1.0]-hexane-6-carboxylic acid methyl ester as described in General Procedure 9. (87% yield)

White solid, mp: 174-176° C. ¹H NMR (D₂O) δ: 7.13 (d, 2 H, J=8.0 Hz), 6.80 (d, 2 H, J=6.8 Hz), 4.79 (m, 1 H), 3.32 (dd, 1 H, J=14.2, 5.4 Hz), 2.96 (dd, 1 H, J=14.2, 10.7), 2.25-2.10 (m, 2 H), 1.92-1.79 (m, 2 H), 1.59-1.41 (m, 3 H). ¹³C NMR (CD₃OD) δ: 173.1, 173.0, 169.5, 156.0, 129.7, 114.9, 65.9, 53.7, 35.2, 31.1, 30.4, 28.9, 24.9, 21.4. MS (Electrospray): 349 (M⁺+1), 332. [α]_{D}²⁵=−47.1° (c=1.1, 1N HCl).

### EXAMPLE 7

### Synthesis of (1S,2S,5R,6S)-2-amino-2-[(2′S)-succinyl]carbamoyl-bicyclo[3.1.0]hexane-6-carboxylic acid hydrochloride

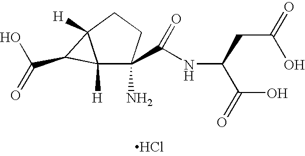

### A. (1S,2S,5R,6S)-2-tert-Butoxycarbonylamino-2-[(2′S)-succinyl dimethyl ester]carbamoyl-bicyclo3.1.0]hexane-6-carboxylic acid 6-methyl ester

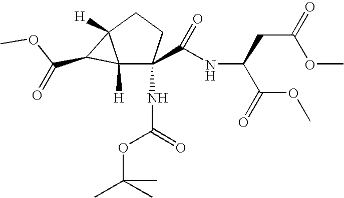

The title compound was prepared from (L)-aspartic acid dimethyl ester as described in General Procedure 3. (87% yield)

Colorless oil.

[α]_{D}²⁵=+24.9° (c=1.0, CHCl₃). ¹H NMR (CDCl₃) δ: 7.45 (brs, 1H), 5.24 (brs, 1H), 4.81 (m, 1H), 3.72 (s, 3H), 3.66 (s, 3H), 3.63 (s, 3H), 3.00 (dd, 1H, J=17.0, 4.6 Hz), 2.85 (dd, 1H, J=17.0, 4.7 Hz), 2.36 (m, 2H), 2.08-1.85 (m, 3H), 1.65 (t, 1H, J=2.8 Hz), 1.40 (s, 9H), 1.13 (s, 1H). ¹³C NMR (CDCl₃) δ: 172.8, 172.7, 171.3, 171.0, 155.0, 74.4, 67.0, 52.6, 51.9, 51.7, 48.7, 36.0, 35.0, 33.0, 28.7, 28.1, 26.8, 21.2.

### B. (1S,2S,5R,6S)-2-Amino-2-[(2′S)-succinyl]carbamoyl-bicyclo[3.1.0]hexane-6-carboxylic acid hydrochloride

The title compound was prepared from (1S,2S,5R,6S)-2-tert-butoxycarbonylamino-2-[(2′S)-succinyl dimethyl ester]carbamoyl-bicyclo[3.1.0]hexane-6-carboxylic acid 6-methyl ester as described in General Procedure 9. (57% yield)

White solid. mp 215° C., dec. [α]_{D}²⁵=−28.4° (c=1.6, MeOH). ¹H NMR (Methanol-d₄) δ: 4.84 (dd, 1H, J=7.8, 5.2 Hz), 3.09 (dd, 1H, J=16.8, 5.2 Hz), 2.92 (dd, 1H, J=16.8, 7.8 Hz), 2.33 (m, 2H), 2.04 (m, 4H), 1.67 (m, 1H). ¹³C NMR (Methanol-d₄) δ: 173.6, 173.4, 172.4, 170.1, 66.5, 49.8, 35.3, 31.5, 31.0, 29.3, 25.6, 21.9.

### EXAMPLE 8

### Synthesis of (1S,2S,5R,6S)-2-amino-2-[(1′S)-carboxy-(2′R)-hydroxypropyl]carbamoyl-bicyclo[3.1.0]hexane-6-carboxylic acid hydrochloride

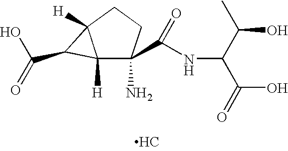

### A. (1S,2S,5R,6S)-2-tert-Butoxycarbonylamino-2-[(1′S)-carboxy-(2′R)-tert-butyldimethylsilyloxyethyl]carbamoyl-bicyclo[3.1.0]hexane-6-carboxylic acid 6-methyl ester.

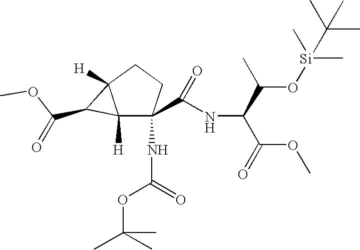

The title compound was prepared from (2S,3S)-2-amino-3-(tert-butyl-dimethyl-silanyloxy)-butyric acid methyl ester (which was prepared as described by Meyers et al., Tetrahedron Lett. 1996, 37, 1743-1746 for the serine analog) as described in General Procedure 3. (90% yield)

Colorless oil. [α]_{D}²⁵=−4.23° (c=1.23, CHCl₃). ¹H NMR (CDCl₃) δ: 7.10 (brs, 1H), 5.36 (brs, 1H), 4.48-4.36 (m, 2H), 3.63 (s, 3H), 3.57 (s, 3H), 2.50 (m, 1H), 2.29-1.79 (m, 4H), 1.63 (t, 1H, J=2.8 Hz), 1.36 (s, 9H), 1.12 (d, 3H, J=6.2 Hz), 0.80 (s, 9H), −0.04 (s, 3H), −0.07 (s, 3H). ¹³C NMR (CDCl₃) δ: 173.2, 172.6, 170.7, 154.9, 80.1, 68.6, 66.9, 57.6, 51.8, 51.4, 34.6, 33.3, 28.8, 28.0, 26.1, 25.4, 20.9, 20.6, −3.80, −4.62.

### B. (1S,2S,5R,6S)-2-amino-2-[(1′S)-carboxy-(2′R)-hydroxypropyl]carbamoyl-bicyclo[3.1.0]hexane-6-carboxylic acid hydrochloride

The title compound was prepared from (1S,2S,5R,6S)-2-tert-butoxycarbonylamino-2-[(1′S)-1′-carboxy-1′-[(1″R)-1″-tert-butyldimethylsiyloxy]ethyl]carbamoyl-bicyclo[3.1.0]hexane-6-carboxylic acid 6-methyl ester as described in General Procedure 9. (26% yield)

White solid. mp 193-194° C. [α]_{D}²⁵=19.8 ° (c=0.5, MeOH). ¹H NMR (D₂O) δ: 4.52 (m, 1H), 4.51-4.43 (m, 1H), 2.41 (m, 2H), 2.17-2.05 (m, 4H), 1.73 (m, 1H), 1.19 (d, 3H, J=6.4 Hz). ¹³C NMR (Methanol-d₄) δ: 175.1, 173.2, 171.1, 66.7, 66.3, 58.4, 31.0, 30.6, 29.2, 25.2, 21.6, 19.1.

### EXAMPLE 9

### Synthesis of (1S,2S,5R,6S)-2-amino-2-[(1′S)-carboxy-2′-hydroxyethyl]carbamoyl-bicyclo[3.1.0]hexane-6-carboxylic acid hydrochloride

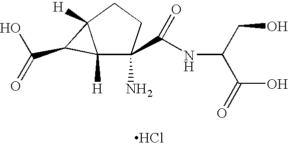

### A. (1S,2S,5R,6S)-2-Amino-2-[(1′S)-carboxy-2′-(tert-butyldimethylsilyloxy)ethyl]carbamoyl-bicyclo[3.1.0]hexane-6-carboxylic acid 6-methyl ester.

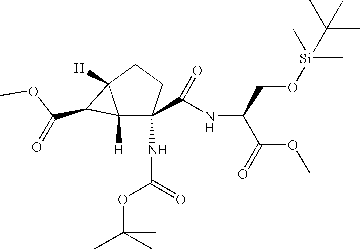

The title compound was prepared from (L)-2-amino-3-(tert-butyl-dimethyl-silanyloxy)-propionic acid methyl ester (which was prepared as described by Meyers et al., Tetrahedron Lett. 1996, 37, 1743-1746) as described in General Procedure 3. (85% yield)

Colorless oil. [α]_{D}²⁵=+20.5° (c=1.23, CHCl₃). ¹H NMR (CDCl₃) δ: 7.26 (brs, 1H), 5.36 (brs, 1H), 4.59-4.52 (m, 1H), 3.98 (dd, 1H, J=10.0, 2.9 Hz), 3.76 (dd, 1H, J=10.0, 3.4 Hz), 3.61 (s, 3H), 3.56 (s, 3H), 2.36 (m, 2H), 2.10-1.78 (m, 3H), 1.63 (t, 1H, J=2.8 Hz), 1.34 (s, 9H), 1.12-1.04 (m, 1H), 0.78 (s, 9H), −0.04 (s, 3H), −0.05 (s, 3H). ¹³C NMR (CDCl₃) δ: 172.7, 172.6, 170.6, 155.0, 80.4, 66.9, 63.2, 54.3, 52.0, 51.5, 35.0, 32.8, 28.6, 28.0, 26.6, 25.5, 21.2, −3.7, −4.0.

### B. (1S,2S,5R,6S)-2-Amino-2-[(1′S)-carboxy-2′-hydroxyethyl]carbamoyl-bicyclo[3.1.0]hexane-6-carboxylic acid hydrochloride

The title compound was prepared from (1S,2S,5R,6S)-2-amino-2-[(1′S)-1′-carboxy-1′-(tert-butyldimethylsilyloxy)methyl]-carbamoyl-bicyclo[3.1.0]hexane-6-carboxylic acid 6-methyl ester as described in General Procedure 9. (20% yield)

White solid. mp 194° C., dec. [α]_{D}²⁵=−5.79° (c=0.5, MeOH). ¹H NMR (Methanol-d₄) δ: 4.56 (brt, 1H, J=4.0 Hz), 3.96 (AB system, 2H), 2.32-2.00 (m, 6H), 1.59 (m, 1H). ¹³C NMR (Methanol-d₄) δ: 173.5, 172.4, 171.8, 66.5, 61.2, 55.5, 31.4, 31.1, 29.1, 25.7, 21.8.

### EXAMPLE 10

### Synthesis of (1S,1′S,2S,5R,6S)-2-amino-2-[(1′S)-carboxy-2-phenylethyl]carbamoyl-bicyclo[3.1.0]hexane-6-carboxylic acid

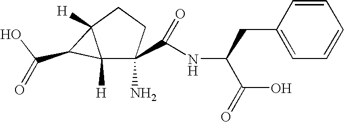

### A. (1S,2S,5R,6S)-2-Allyloxycarbonylamino-2-[(1′S)-allyloxycarbonyl-2′-phenylethyl]carbamoyl-bicyclo[3.1.0]hexane-6-carboxylic acid allyl ester

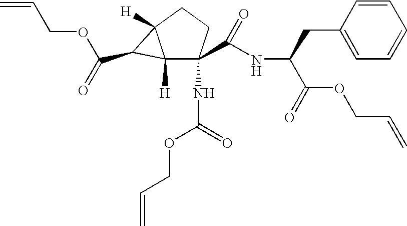

The title compound was prepared from (L)-phenyl alanine allyl ester as described in General Procedure 1. (79% yield)

White solid. ¹H NMR (CDCl₃) δ: 7.27-7.08 (m, 5 H); 7.01-6.97 (bd, 1 H); 5.96-5.75 (m, 3 H); 5.64 (bs, 1 H); 5.32-5.17 (m, 6 H); 4.83 (c, J=6.4 Hz, 1 H); 4.59-4.49 (m, 6 H); 3.17-3.09 (m, 2 H); 2.42 (bs, 1 H); 2.30 (dd, J=13.2, 7.5 Hz, 1 H); 2.03-1.84 (m, 3 H), 1.66 (t, J=3.0 Hz, 1H); 1.24-1.08 (m, 1H).

### B. (1S,2S,5R,6S)-2-Amino-2-[(1′S)-carboxy-2′-phenyl-ethyl]carbamoyl-bicyclo[3.1.0]hexane-6-carboxylic acid

The title compound was prepared from (1S,1′S,2S,5R,6S)-2-allyloxycarbonylamino-2-(1′S-allyloxycarbonyl-2′-phenyl-ethylcarbamoyl)-bicyclo[3.1.0]hexane-6-carboxylic acid allyl ester as described in General Procedure 8. The solid was stirred in MeOH for 15 min and filtered. (48% yield)

White solid. ¹H-NMR (D₂O)δ: 7.07-7.04 (m, 5 H); 4.35-4.32 (m, 1 H); 3.15-3.08 (m, 1 H); 2.85-2.74 (m, 1 H); 1.88 (m, 1 H); 1.74-1.74 (m, 1 H); 1.52-1.10 (m, 5 H). ¹³C-NMR (DMSO-d₆)δ: 174.0, 173.3, 171.6, 138.5, 129.7 (2C), 128.3 (2C), 126.5, 65.6, 54.7, 37.0, 33.8, 32.8, 28.6, 26.1, 21.7.

### EXAMPLE 11

### Synthesis of (1S,2S,5R,6S)-2-amino-2-[(1′S)-carboxy-2′-methylpropyl]carbamoyl-bicyclo[3.1.0]hexane-6-carboxylic acid

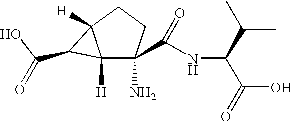

### A. (1S,2S,5R,6S)-2-Allyloxycarbonylamino-2-[(1′S)-allyloxycarbonyl-2′-methylpropyl]carbamoyl-bicyclo[3.1.0]hexane-6-carboxylic acid allyl ester

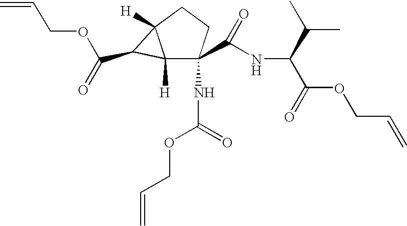

The title compound was prepared from (L)-valine allyl ester as described in General Procedure 1. (82% yield)

White solid. ¹H-NMR (CDCl₃)δ: 5.88 (m, 3H); 5.49-5.17 (m, 7H); 4.64-4.51 (m, 7H); 2.55 (bs, 1H); 2.43 (dd, J=13.2, 8.3 Hz, 1H); 2.30-1.87 (m, 4H); 1.71 (t, J=2.7 Hz, 1H); 1.29-1.13 (m, 1H); 0.96 (d, J=6.7 Hz, 3H); 0.90 (d, J=7.0 Hz, 3H).

### B. (1S,2S,5R,6S)-2-Amino-2-[(1′S)-carboxy-2′-methyl-propyl]carbamoyl-bicyclo[3.1.0]hexane-6-carboxylic acid

The title compound was prepared from (1S,2S,5R,6S)-2-allyloxycarbonylamino-2-[(1′S)-allyloxycarbonyl-2′-methyl-propylcarbamoyl]-bicyclo[3.1.0]hexane-6-carboxylic acid allyl ester as described in General Procedure 8. The solid was stirred with MeOH for 15 min and filtered. (56% yield)

White solid. ¹H-NMR (DMSO-d₆)δ: 4.05 (m, 1 H); 2.18-1.85 (m, 7 H); 1.28-1.17 (m, 1 H); 0.86 (d, J=6.4 Hz, 3 H); 0.84 (d, J=6.5 Hz, 3 H). ¹³C-NMR (DMSO-d₆)δ: 174.2, 173.4, 173.2, 65.7, 58.1, 35.4, 33.3, 30.9, 28.7, 26.8, 21.4, 19.7, 18.3.

### EXAMPLE 12

### Synthesis of (1S,2S,5R,6S)-2-amino-2-[(1S)-carboxy-(2′R)-methylbutyl]carbamoyl-bicyclo[3.1.0]hexane-6-carboxylic acid

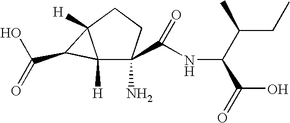

### A. (1S,2S,5R,6S)-2-Allyloxycarbonylamino-2-[(1′S)-allyloxycarbonyl-(2′R)-methylbutyl]carbamoyl-bicyclo[3.1.0]hexane-6-carboxylic acid allyl ester

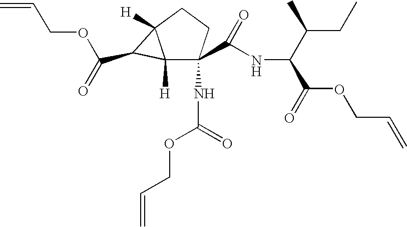

The title compound was prepared from (L)-isoleucine allyl ester as described in General Procedure 1. (72% yield)

White solid. 1H-NMR (CDCl₃)δ: 5.90-5.87 (m, 3 H); 5.53 (bs, 1 H); 5.37-5.16 (m, 6 H); 4.63-4.52 (m, 7 H); 2.53 (bs, 1 H); 2.40 (dd, J=13.4, 8.3 Hz, 1 H); 2.12-1.87 (m, 4 H); 1.70 (t, J=3.0 Hz, 1 H); 1.48-1.05 (m, 3 H); 0.90 (t, J=7.3 Hz, 3 H); 0.90 (d, J=7.0 Hz, 3 H).

### B. (1S,2S,5R,6S)-2-amino-2-[(1′S)-carboxy-(2′R)-methylbutyl]carbamoyl-bicyclo[3.1.0]hexane-6-carboxylic acid

The reaction was preformed using (1S,2S,5R,6S)-2-allyloxycarbonylamino-2-(1′S-allyloxycarbonyl-2′-methyl-butylcarbamoyl)-bicyclo[3.1.0]hexane-6-carboxylic acid allyl ester as described in General Procedure 8. The residue was purified by cation exchange chromatography, eluting with 2N ammonia in methanol. The yellow solid obtained was stirred with methanol for 15 min, filtered and dried to provide the title compound. (31% yield)

White solid. ¹H-NMR (DMSO-d₆)δ: 4.03 (bs, 1 H); 1.82 (m, 7 H); 1.54-1.38 (m, 1 H); 1.29-1.02 (m, 2 H); 0.87-0.80 (m, 6 H). ¹³C-NMR (DMSO-d₆)δ: 174.4, 173.5, 173.2, 65.7, 57.6, 37.7, 35.7, 33.7, 28.5, 27.0, 25.2, 21.6, 16.1, 12.0

### EXAMPLE 13

### Synthesis of (1S,2S,5R,6S)-2-amino-2-[(1′S)-carboxy-ethyl]carbamoyl-bicyclo[3.1.0]hexane-6-carboxylic acid

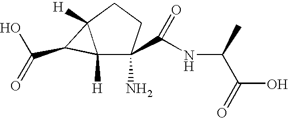

### A. (1S,2S,5R,6S)-2-Allyloxycarbonylamino-2-[(1′S)-ethoxycarbonylethyl]carbamoyl bicyclo[3.1.0]hexane-6-carboxylic acid allyl ester

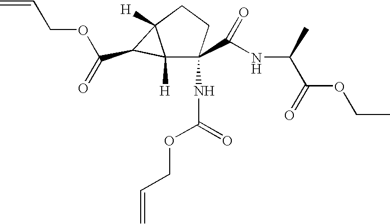

The title compound was prepared from (L)-alanine ethyl ester as described in General Procedure 1. (92% yield) The crude was directly used for the next step.

### B. (1S,2S,5R,6S)-2-Amino-2-[(1′S)-carboxyethyl]carbamoy-bicyclo[3.1.0]hexane-6-carboxylic acid

The reaction was preformed using (1S,2S,5R,6S)-2-allyloxycarbonylamino-2-[(1′S)-ethoxycarbonyl-ethyl]carbamoyl-bicyclo[3.1.0]hexane-6-carboxylic acid allyl ester as described in General Procedure 8. The residue was suspended in 2 mL of THF and 4 mL of 2.5N LiOH was added. The reaction was stirred for 2 h, acidified to pH 3 with 6N HCl and evaporated to dryness. Purification by ion exchange chromatography provided the title compound. (38% yield)

White solid. ¹H-NMR (D₂O)δ: 4.12 (q, J=7.3 Hz, 1 H); 2.14-2.13 (m, 2 H); 2.00-1.90 (m, 3 H); 1.79 (t, J=3.0 Hz, 1 H); 1.59-1.41 (m, 1 H); 1.25 (d, J=7.3 Hz, 3 H). ¹³C-NMR (D₂O)δ: 174.8, 177.2, 170.1, 66.1, 50.8, 30.9, 29.7, 28.4, 24.9, 22.8, 16.8.

### EXAMPLE 14

### Synthesis of (1S,2S,5R,6S)-2-amino-2-[(1′S)-carboxyphenyl-methyl]carbamoyl-bicyclo[3.1.0]hexane-6-carboxylic acid

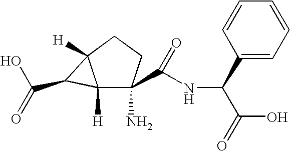

### A. (1S,2S,5R,6S)-2-Allyloxycarbonylamino-2-[(1′S)-methoxycarbonylphenylmethyl]carbamoyl-bicyclo[3.1.0]hexane-6-carboxylic acid allyl ester

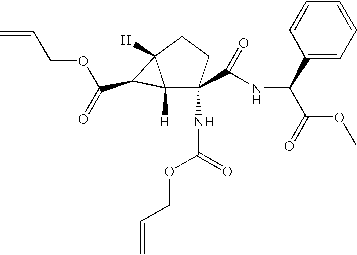

The title compound was prepared from (S)-phenylglycine methyl ester as described in General Procedure 1. (65% yield)

White solid. ¹H-NMR (CDCl₃)δ: 7.62 (s, 1 H); 7.31-7.31 (m, 5 H); 5.96-5.77 (m, 2 H); 5.67 (s, 1 H); 5.52 (d, J=7.0 Hz, 1 H); 5.31-0.00 (m, 4 H); 4.52 (dt, J=5.6, 1.3 Hz, 4 H); 3.68 (s, 3 H); 2.48-2.37 (m, 2 H); 2.13-1.85 (m, 4 H); 1.71 (t, J=3.0 Hz, 1 H); 1.26-1.18 (m, 1 H).

### B. (1S,2S,5R,6S)-2-amino-2-[(1′S)-carboxyphenyl-methyl]carbamoyl-bicyclo[3.1.0]hexane-6-carboxylic acid

The reaction was performed using (1S,2S,5R,6S)-2-allyloxycarbonylamino-2-[(1′S)-methoxycarbonylphenymethyl]carbamoyl-bicyclo[3.1.0]hexane-6-carboxylic acid allyl ester, as described in General Procedure 8. The residue was suspended in 4 mL of tetrahydrofuran and 9 mL of 2.5 N lithium hydroxide was added. The reaction was stirred for 2 h, acidified to pH 3 with 6N HCl and evaporated to dryness. Purification by ion exchange chromatography provided the title compound. (47% yield)

White solid. ¹H-NMR (D₂O+Py-d₅)δ: 7.34 (s, 5 H); 5.09 (s, 1 H); 2.18-1.78 (m, 6 H); 1.65-1.54 (m, 1 H). ¹³C-NMR (D₂O+Py-d₅)δ: 177.9, 174.3, 168.9, 137.4, 127.9, 127.0, 125.8, 65.2, 58.6, 29.9, 28.6, 26.4, 24.4, 23.5.

### Example 15

### Synthesis of (1S,2S,5R,6S) 2-Amino-6-[(1′S)-carboxy-2′-phenylethyl]carbamoyl-bicyclo[3.1.0]hexane-2-carboxylic acid hydrochloride

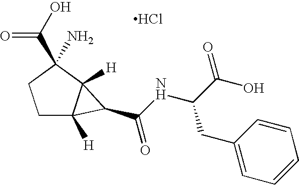

### A. (1S,2S,5R,6S)-3-allyloxycarbonyl-5-oxo-4-spiro-6′-[(1″S)-carboxy-2″-phenylethyl]carbamoyl-2″(bicyclo[3,1,0]-hexane) oxazolidine

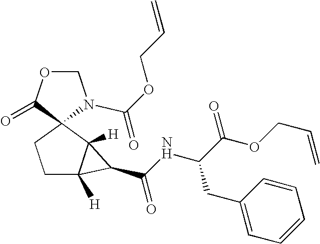

The title compound was prepared from (L)-phenylalanine allyl ester as described in General Procedure 5. (47 %)

¹H-NMR (CDCl₃)δ: 7.33-7.24 (m, 3 H); 7.15-7.10 (m, 2 H); 6.17 (d, J=7.8 Hz, 1 H); 6.00-5.75 (m, 2 H); 5.34-5.18 (m, 6 H); 4.87 (dt, J=8.1, 5.9 Hz, 1 H); 4.61-4.57 (m, 4 H); 3.12 (d, J=6.2 Hz, 2 H); 2.43-2.15 (m, 3 H); 2.07-1.86 (m, 3 H); 1.77-1.70 (m, 1 H).

### B. (1S,2S,5R,6S)-2-Amino-6-[(1′S)-carboxy-2′-phenyl-ethyl]carbamoyl-bicyclo[3.1.0]hexane-2-carboxylic acid hydrochloride

The title compound was prepared from (1S,2S,5R,6S)-3-allyloxycarbonyl-5-oxo-4-spiro-6′-((1S)-1-carboxy-2-phenyl-ethylcarbamoyl)-2′(bicyclo[3,1,0]-hexane)oxazolidine as described in General Procedure 6. (91 %)

¹H-NMR (CD₃OD)δ: 7.33-7.23 (m, 5 H); 4.71 (dd, J=8.41, 5.40 Hz, 1 H); 3.22 (dd, J=13.81, 5.20 Hz, 1 H); 3.00 (dd, J=14.01, 8.41 Hz, 1 H); 2.21-1.86 (m, 6 H); 1.58-1.43 (m, 1 H). ¹³C-NMR (CD₃OD)δ: 173.8, 171.5, 171.3, 128.9 128.0, 126.4, 65.4, 53.6, 37.1, 31.4, 30.1, 27.4, 26.3, 22.2.

1. A compound of the formula I 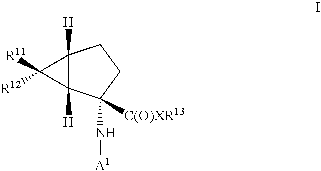
wherein
R¹¹ is C(O)YR¹⁴ and R¹² is hydrogen or fluoro; or R¹¹ is hydrogen or fluoro and R¹² is C(O)YR¹⁴;
R¹³ and R¹⁴ are, independently, hydrogen, (1-10C) alkyl, (2-4C) alkenyl, (2-4C) alkynyl, or aryl;
A¹ is hydrogen or an amino acyl bonded through the carbonyl to form an amine terminus;
X and Y are, independently, O or A²;
A² is an amino acyl bonded through the amine to form a carboxlyate terminus;
provided when x is O, Y is not O;
or a pharmaceutically acceptable salt thereof.

2. A compound of the formula I 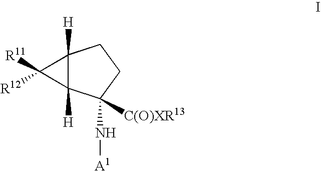
wherein
R¹¹ is C(O)YR¹⁴ and R¹² is hydrogen or fluoro; or R¹¹ is hydrogen or fluoro and R¹² is C(O)YR¹⁴;
R¹³ and R¹⁴ are, independently, hydrogen, (1-10C) alkyl, (2-4C) alkenyl, (2-4C) alkynyl, or aryl;
A¹ is hydrogen or an α-amino acyl bonded through the carbonyl to form an amine terminus;
X and Y are independently O or A²;
A² is an α-amino acyl bonded through the amine to form a carboxlyate terminus;
provided when X is O, Y is not O;
or a pharmaceutically acceptable salt thereof.

3. A compound of the formula I 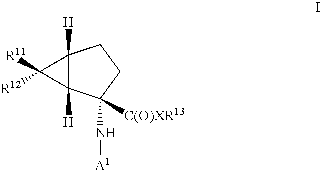
wherein
R¹¹ is C(O)YR¹⁴ and R¹² is hydrogen or fluoro; or R¹¹ is hydrogen or fluoro and R¹² is C(O)YR¹⁴;
R¹³ and R¹⁴ are, independently, hydrogen, (1-10C) alkyl, (2-4C) alkenyl, (2-4C) alkynyl, or aryl;
A¹ is hydrogen;
X and Y are, independently, O or A²;
A² is an amino acyl bonded through the amine to form a carboxlyate terminus;
provided when X is O, Y is not O;
or a pharmaceutically acceptable salt thereof.

4. A compound of the formula I 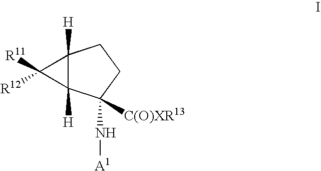
wherein
R¹¹is C(O)YR¹⁴ and R¹² is hydrogen or fluoro; or R¹¹is hydrogen or fluoro and R¹² is C(O)YR¹⁴;
R¹³ and R¹⁴ are, independently, hydrogen, (1-10C) alkyl, (2-4C) alkenyl, (2-4C) alkynyl, or aryl;
A¹ is hydrogen;
X and Y are independently O or A²;
A² is an α-amino acyl bonded through the amine to form a carboxlyate terminus;
provided when X is O, Y is not O;
or a pharmaceutically acceptable salt thereof.

5. The compound (or salt thereof) of claims 1-4 wherein (1-10C) alkyl is methyl.

6. The compound (or salt thereof) of claims 1-5 wherein amino acyl is independently selected from L-alanyl, glycyl, L-leucyl, L-phenylalanyl, L-valyl, L-isoleucyl, L-methionyl, L-tyrosyl, L-aspartyl, L-prolyl, L-serinyl, D-phenylglycyl, L-phenylglycyl, L-asparagyl and L-threonyl.

7. The compound (or salt thereof) of claims 1-6 wherein
X is A²;
Y is O;
R¹¹ is C(O)YR¹³; and
R¹², R¹³, and R¹⁴ are hydrogen.

8. The compound (or salt thereof) of claim 7 wherein A² is L-valyl.

9. The compound (or salt thereof) of claims 1-4 or 6 wherein
Y is A²;
X is O;
R¹¹is C(O)YR¹⁴;
R¹², R¹⁴, and R¹³ are hydrogen; and
A² is L-phenylalanyl.

10. The compound of claim 1 which is selected from the group consisting of (1S,2S,5R,6S) 2-amino-2-[(1′S)-carboxy-3′-methylbutyl]carbamoyl-bicyclo[3.1.0]hexane-6-carboxylic acid, 1S,1′S,2S,5R,6S)-2-amino-2-(carboxymethyl-carbamoyl)-bicyclo[3.1.0]hexane-6-carboxylic acid, (1S,2S,5R,6S)-2-amino-2-[(1′R)-carboxy-phenylmethyl]carbamoyl-bicyclo[3.1.0]hexane-6-carboxylic acid, (1S,2S,5R,6S)-2-amino-2-[(1′S)-carboxy-3′-methylthiopropyl]carbamoyl-bicyclo[3.1.0]hexane-6-carboxylic acid, (1′S,2′S,5′R,6′S)-2′-amino-6′-carboxy-bicyclo[3.1.0]-hexane-2′-carbonyl-pyrrolidine-2S carboxylic acid, (1S,2S,5R,6S)-2-amino-2-[1′S-carboxy-2′-(4″-hydroxyphenyl)ethyl]carbamoyl-bicyclo[3.1.0]-hexane-6-carboxylic acid, (1S,2S,5R,6S)-2-amino-2-[(2′S)-succinyl]carbamoyl-bicyclo[3.1.0]hexane-6-carboxylic acid hydrochloride, (1S,2S,5R,6S)-2-amino-2-[(1′S)-carboxy-(2′R)-hydroxypropyl] carbamoyl-bicyclo[3.1.0]hexane-6-carboxylic acid hydrochloride, (1S,2S,5R,6S)-2-amino-2-[(1′S)-carboxy-2′-hydroxyethyl]carbamoyl-bicyclo[3.1.0]hexane-6-carboxylic acid hydrochloride, (1S,1′S,2S,5R,6S)-2-amino-2-[(1′S)-carboxy-2-phenylethyl]carbamoyl-bicyclo[3.1.0]hexane-6-carboxylic acid, (1S,2S,5R,6S)-2-amino-2-[(1′S)-carboxy-2′-methylpropyl]carbamoyl-bicyclo[3.1.0]hexane-6-carboxylic acid, (1S,2S,5R,65)-2-amino-2-[(1′S)-carboxy-(2′R)-methylbutyl]carbamoyl-bicyclo[3.1.0]hexane-6-carboxylic acid, (1S,2S,5R,6S)-2-amino-2-[(1′S)-carboxy-ethyl]carbamoyl-bicyclo[3.1.0]hexane-6-carboxylic acid, (1S,2S,5R,6S) -2-amino-2-[(1′S)-carboxyphenyl-methyl]carbamoyl-bicyclo[3.1.0]hexane-6-carboxylic acid, (1S,2S,5R,6S) 2-Amino-6-[(1′S)-carboxy-2′-phenylethyl]carbamoyl-bicyclo[3.1.0]hexane-2-carboxylic acid hydrochloride.

11. A pharmaceutically acceptable salt of a compound of formula I as claimed in claims 1-10 which is an acid-addition salt made with an acid which provides a pharmaceutically acceptable anion or, for a compound which contains an acidic moiety, which is a salt made with a base which provides a pharmaceutically acceptable cation.

12. A pharmaceutical formulation comprising in association with a pharmaceutically acceptable carrier, dilutent or excipient, a compound of formula I (or a pharmaceutically acceptable salt thereof) as provided in claims 1-11.

13. A process for preparing the compound of formula I, or a pharmaceutically acceptable salt thereof, as claimed in claim 1 which is selected from:
(A) for a compound of formula I in which R¹¹ is carboxy and X is A², deprotecting the amine group of a compound of formula III 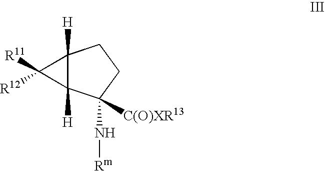
in which R^{m} is an amine-protecting group;
(B) for a compound of formula I in which R¹¹ is a carboxy and X is A², deprotecting the amine and carboxy groups of a compound of formula III in which R^{m} is an amine-protecting group and R¹⁴ is a carboxy protecting group;
(C) for a compound of formula I in which Y is A² and X is O and R¹³ is hydrogen, deprotecting and ring-opening a compound of formula IV; 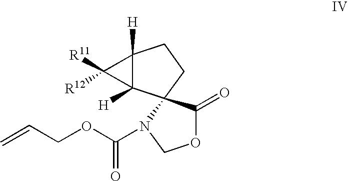
whereafter, for any of the above procedures, when a functional group is protected using a protecting group, removing the protecting group;
whereafter, for any of the above procedures, when a pharmaceutically acceptable salt of a compound of formula I is required, it is obtained by reacting the basic form of such a compound of formula I with an acid affording a physiologically acceptable counterion, or, for a compound of formula I which bears an acidic moiety, reacting the acidic form of such a compound of formula I with a base which affords a pharmaceutically acceptable cation, or by any other conventional procedure.

14. A method for affecting the cAMP-linked metabotropic glutamate receptors in a patient, which comprises administering to a patient requiring modulated excitatory amino acid neurotransmission a pharmaceutically-effective amount of a compound of claim 1.

15. A method for affecting the cAMP-linked metabotropic glutamate receptors in a patient, which comprises administering to a patient requiring modulated excitatory amino acid neurotransmission a pharmaceutically-effective amount of a compound of claim 10.

16. A method for treating a neurological disorder in a patient which comprises administering to the patient in need of treatment thereof a pharmaceutically-effective amount of a compound of claim 1.

17. The method of claim 16 wherein said neurological disorder is cerebral deficits subsequent to cardiac bypass and grafting; cerebral ischemia; spinal cord trauma; head trauma; Alzheimer's Disease; Huntington's Chorea; amyotrophic lateral sclerosis; AIDS-induced dementia; perinatal hypoxia; hypoglycemic neuronal damage; ocular damage and retinopathy; cognitive disorders; idiopathic and drug-induced Parkinson's Disease; muscular spasms; migraine headaches; urinary incontinence; drug tolerance, withdrawal, and cessation; smoking cessation; emesis; brain edema; chronic pain; sleep disorders; convulsions; Tourette's syndrome; attention deficit disorder; and tardive dyskinesia.

18. The method of claim 17 wherein said neurological disorder is drug tolerance, withdrawal, and cessation; or smoking cessation.

19. A method for treating a neurological disorder in a patient which comprises administering to the patient in need of treatment thereof a pharmaceutically-effective amount of a compound of claim 10.

20. The method of claim 19 wherein said neurological disorder is cerebral deficits subsequent to cardiac bypass and grafting; cerebral ischemia; spinal cord trauma; head trauma; Alzheimer's Disease; Huntington's Chorea; amyotrophic lateral sclerosis; AIDS-induced dementia; perinatal hypoxia; hypoglycemic neuronal damage; ocular damage and retinopathy; cognitive disorders; idiopathic and drug-induced Parkinson's Disease; muscular spasms; migraine headaches; urinary incontinence; drug tolerance, withdrawal, and cessation; smoking cessation; emesis; brain edema; chronic pain; sleep disorders; convulsions; Tourette's syndrome; attention deficit disorder; and tardive dyskinesia.

21. The method of claim 20 wherein said neurological disorder is drug tolerance, withdrawal, and cessation; or smoking cessation.

22. A method for treating a psychiatric disorder in a patient which comprises administering to the patient in need of treatment thereof a pharmaceutically-effective amount of a compound of claim 1.

23. The method of claim 22 wherein said psychiatric disorder is schizophrenia, anxiety and related disorders such as GAD or panick attack, depression, bipolar disorders, psychosis, and obsessive compulsive disorders.

24. The method of claim 23 wherein said psychiatric disorder is anxiety and related disorders, such as GAD or panick attack.

25. A method for treating a psychiatric disorder in a patient which comprises administering to the patient in need of treatment thereof a pharmaceutically-effective amount of a compound of claim 10.

26. The method of claim 25 wherein said psychiatric disorder is schizophrenia, anxiety and related disorders such as GAD or panic attack, depression, bipolar disorders, psychosis, and obsessive compulsive disorders.

27. The method of claim 26 wherein said psychiatric disorder is anxiety and related disorders such as GAD or panic attack.

28. A method of administering an effective amount of a compound of formula II, where R¹³ and R¹⁴ are both hydrogen (a di-acid), which comprises administering to a patient requiring modulated excitatory amino acid neurotransmission a pharmaceutically effective amount of a compound of claim 1.

29. A method for affecting the CAMP-linked metabotropic glutamate receptors in a mammal, which comprises administering to a mammal requiring modulated excitatory amino acid neurotransmission a pharmaceutically effective amount of a compound of formula I substantially as hereinbefore described with reference to any of the Examples.

30. A process for preparing a novel compound of formula I substantially as hereinbefore described with reference to any of the Examples.
